(19) United States
(12) Patent Application Publication
(10) Pub. No.: US 2024/0325413 A1
(43) Pub. Date: Oct. 3, 2024

(54) FORMULATIONS FOR THE TREATMENT OF RESPIRATORY DISORDERS

(71) Applicant: 484 Science Corp, Richmond Hill (CA)

(72) Inventors: Steven Ellis, Oro-Medonte (CA); Bildad Kimani Nyambura, Reading (GB); Alex Stenzler, Long Beach, CA (US)

(21) Appl. No.: 18/577,390

(22) PCT Filed: Jul. 7, 2022
(86) PCT No.: PCT/US2022/073496

§ 371 Date: Jan. 8, 2024

Related U.S. Application Data
(60) Provisional application No. 63/219,499, filed on Jul. 8, 2021.

(51) Int. Cl. A61K 31/00; A61K 9/00; A61K 31/40; A61K 31/4704; A61K 45/06; A61K 47/06

(52) U.S. Cl. CPC A61K 31/658; A61K 9/0075; A61K 9/008; A61K 31/40; A61K 31/4704; A61K 45/06; A61K 47/06

(57) ABSTRACT

The present invention provides formulations for inhalation comprising at least one cannabinoid and at least one additional therapeutic agent selected from the group consisting of a beta-2 agonist and a muscarinic antagonist. The formulation may be a pMDI formulation further comprising a solvent, a propellant, a co-solvent, a surfactant, and/or a stabilizer, or a dry powder formulation further comprising an excipient. The formulation may be useful in the treatment of obstructive or inflammatory airways diseases.

### CROSS-REFERENCE TO RELATED APPLICATIONS

The present application is a 35 U.S.C. § 371 national phase application from, and claiming priority to, International Application PCT/US2022/073496, filed Jul. 7, 2022, which claims priority to U.S. Provisional Application No. 63/219,499, filed Jul. 8, 2021, each of which is incorporated by reference herein in its entirety.

### BACKGROUND OF THE INVENTION

Respiratory disorders are often treated with corticosteroids, β agonists, Muscarinic agonists, and combinations thereof. Steroids such as glucocorticoids are useful in the management of many inflammatory diseases such as asthma and COPD for more than 50 years. Regardless of the route used, it is recommended to limit the use of these molecules to patients who require them and to take all possible precautions to minimize side effects. As an example, steroids are known to impact mineral bone density. Therefore, a workable alternative to corticosteroids would be of major benefit to the treatment of respiratory disorders.

There is a need in the art for improved formulations for treatment of respiratory disorders. The present invention meets this need.

### SUMMARY OF THE INVENTION

In one aspect, the present invention relates to a formulation suitable for inhalation, the formulation comprising at least one cannabinoid and at least one additional therapeutic agent selected from the group consisting of a beta-2 agonist and a muscarinic antagonist. In one embodiment, the formulation comprises a beta-2 agonist and a muscarinic antagonist. In one embodiment, the formulation does not comprise a corticosteroid. In one embodiment, the formulation comprises a flavor component. In one embodiment, the formulation comprises a cough suppressant.

In one embodiment, the at least one cannabinoid is a member of a class selected from the group consisting of cannabichromenes, cannabicyclols, cannabidiols, cannabielsoins, cannabigerols, cannabinols, cannabinodiols, cannabitriols, cannabichromanones, isocannabinoids, and any stereoisomers, ethers, derivatives, metabolites, esters, salts, or carbon chain homologs thereof. In one embodiment, the formulation comprises at least one cannabinoid selected from the group consisting of cannabidiol, cannabigerol, cannabinol, cannabidivarin, cannabichromene, and any stereoisomers, ethers, derivatives, metabolites, esters, salts, or carbon chain homologs thereof. In one embodiment, the formulation does not comprise a psychoactive cannabinoid. In one embodiment, the formulation does not comprise delta-9-tetrahydrocannabinol.

In one embodiment, the formulation comprises a short-acting beta-2 agonist selected from the group consisting of bitolterol, fenoterol, isoproterenol, levalbuterol, metaproterenol, mabuterol, pirbuterol, procaterol, ritodrine, albuterol, and terbutaline. In one embodiment, the formulation comprises a long-acting beta-2 agonist selected from the group consisting of arformoterol, bambuterol, clenbuterol, formoterol, salmeterol, abediterol, carmoterol, indacaterol, olodaterol, and vilanterol. In one embodiment, the formulation comprises a muscarinic antagonist selected from the group consisting of tiotropium, ipratropium, glycopyrronium, oxitropium, aclidinium, trospium, % umeclidinium, and salts thereof.

In another aspect, the present invention relates to a pressurized metered dose inhaler (pMDI) formulation comprising: at least one cannabinoid; at least one additional therapeutic agent selected from the group consisting of a beta-2 agonist and a muscarinic antagonist; a solvent; and a propellant. In one embodiment, the pMDI formulation does not include delta-9-tetrahydrocannabinol. In one embodiment, the pMDI formulation comprises a cannabinoid disclosed herein. In one embodiment, the pMDI formulation comprises an additional therapeutic disclosed herein. In one embodiment, the propellant is selected from the group consisting of HFA 134a, HFA 227ea, HFA-152a, and HFA-32. In one embodiment, the solvent is selected from the group consisting of water, buffered water, ethanol, propylene glycol, polyethylene glycol, erythritol, xylitol, mannitol, sorbitol, diethylene glycol monoethyl ether (Transcutol), and glycerol. In one embodiment, the pMDI formulation further comprises a surfactant. In one embodiment, the surfactant is selected from the group consisting of Span® 85, polyvinylpyrrolidone, oleic acid, sunflower oil, lecithin, phosphatidylcholine, isopropyl myristate, stearic acid, medium and long chain triglycerides, polysorbate 20, polysorbate 80, and sorbitan trioleate.

In another aspect, the present invention relates to a dry powder formulation comprising: at least one cannabinoid; at least one additional therapeutic agent selected from the group consisting of a beta-2 agonist and a muscarinic antagonist; and an excipient; wherein the dry powder formulation is a dry powder suitable for inhalation. In one embodiment, the dry powder formulation comprises any cannabinoid and/or additional therapeutic agent described herein. In one embodiment, the excipient is selected from the group consisting of an inhalable sugar, a cellulose, a glycol, and an amino acid.

### BRIEF DESCRIPTION OF THE DRAWINGS

The following detailed description of preferred embodiments of the invention will be better understood when read in conjunction with the appended drawings. It should be understood that the invention is not limited to the precise arrangements and instrumentalities of the embodiments shown in the drawings.

FIG. 1 depicts a typical standard chromatogram for an exemplary cannabinoid mixture.

FIG. 2 depicts a typical standard chromatogram for an exemplary mixture of a beta-2 agonist and a muscarinic antagonist.

FIG. 3 depicts typical sample chromatogram for an exemplary formulation.

FIG. 4 is a tabulation of results from NGI testing of Formulation 1.

FIG. 5 is a chart summarizing the NGI distribution of the exemplary mixture of a beta-2 agonist and a muscarinic antagonist of Formulation 1.

FIG. 6 is a chart summarizing the NGI distribution of the exemplary mixture of cannabinoids of Formulation 1.

FIG. 7 is a tabulation of results from NGI testing of Formulation 2.

FIG. 8 is a chart summarizing the NGI distribution of the exemplary mixture of a beta-2 agonist and a muscarinic antagonist of Formulation 2.

FIG. 9 is a chart summarizing the NGI distribution of the exemplary mixture of cannabinoids of Formulation 2.

FIG. 10 is a tabulation of results from NGI testing of Formulation 3.

FIG. 11 is a chart summarizing the NGI distribution of the exemplary mixture of a beta-2 agonist and a muscarinic antagonist of Formulation 3.

FIG. 12 is a chart summarizing the NGI distribution of the exemplary mixture of cannabinoids of Formulation 3.

FIG. 13 is a tabulation of results from NGI testing of Formulation 4.

FIG. 14 is a chart summarizing the NGI distribution of the exemplary mixture of a beta-2 agonist and a muscarinic antagonist of Formulation 4.

FIG. 15 is a chart summarizing the NGI distribution of the exemplary mixture of cannabinoids of Formulation 4.

### DETAILED DESCRIPTION

The present invention provides formulations comprising at least one cannabinoid and at least one additional therapeutic agent selected from the group consisting of a beta-2 agonist therapeutic agent and a muscarinic antagonist therapeutic agent. The invention also relates to, methods for using the same, and methods for making the same. The formulations may further comprise excipients, additional therapeutic agents, and flavor components. The formulations may be manufactured by dry processes and wet processes.

### Definitions

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

As used herein, the term “composition” refers to a mixture of at least one compound or molecule useful within the invention with one or more different compound, molecule, or material.

As used herein the term “formulation amount” refers to the total or partial amount of a formulation packed in a disposable container, or to the total or partial amount of a bulk formulation that can be loaded into a delivery chamber or compartment of a dispenser for a therapeutic agent.

As used herein the term “inhalation” refers to the act of inhaling an amount of a formulation, and can mean for example a single inhalation, or multiple inhalations.

As used herein, an “instructional material” includes a physical or electronic publication, a recording, a diagram, or any other medium of expression which can be used to communicate the usefulness of the composition and method of the invention for its designated use. The instructional material of the kit of the invention may, for example, be affixed to a container which contains the composition or be shipped together with a container which contains the composition. Alternatively, the instructional material may be delivered separately from the container with the intention that the instructional material and the composition be used cooperatively by the recipient.

The term “pharmaceutically acceptable” refers to those properties and/or substances that are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable” may also refer to a carrier, meaning a medium that does not interfere with the effectiveness of the biological activity of the active ingredient(s) and is not toxic to the host to which it is administered. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

Unless stated otherwise, the described size or size range of a particle should be considered as the mass median aerodynamic diameter (MMAD) of the particle or set of particles. Such values are based on the distribution of the aerodynamic particle diameters defined as the diameter of a sphere with a density of 1 gm/cm³ that has the same aerodynamic behavior as the particle which is being characterized. Because any particles described herein may be in a variety of densities and shapes, the size of the particles is expressed as the MMAD and not the actual diameter of the particles.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range.

### Therapeutic Compositions

In one aspect, the present invention relates to a formulation for inhalation, the formulation comprising at least one cannabinoid and at least one additional therapeutic agent selected from the group consisting of a beta-2 agonist and a muscarinic antagonist. In some embodiments, the formulation comprises a beta-2 agonist and a muscarinic antagonist.

The term “cannabinoid” as used herein denotes a class of diverse chemical compounds that act on cannabinoid receptors on cells that repress neurotransmitter release in the brain. The class includes endocannabinoids, phytocannabinoids, and synthetic cannabinoids. Exemplary cannabinoid classes and compounds include, but are not limited to, cannabichromenes such as cannabichromene (CBC), cannabichromenic acid (CBCA), cannabichromevarin (CBCV), and cannabichromevarinic acid (CBCVA); cannabicyclols such as cannabicyclol (CBL), cannabicyclolic acid (CBLA), and cannabicyclovarin (CBLV); cannabidiols such as cannabidiol (CBD), cannabidiol monomethylether (CBDM), cannabidiolic acid (CBDA), cannabidiorcol (CBD-C1), cannabidivarin (CBDV), and cannabidivarinic acid (CBDVA); cannabielsoins such as cannabielsoin (CBE), C3-cannabielsoin (CBE-C3), cannabielsoic acid B (CBEA-B), C3-cannabielsoic acid B (CBEA-C3-B), and cannabielsoin acid A (CBEA-A); cannabigerols such as cannabigerol (CBG), cannabigerol monomethylether (CBGM), cannabigerolic acid (CBGA), cannabigerolic acid monomethylether (CBGAM), cannabigerovarin (CBGV), and cannabigerovarinic acid (CBGVA); cannabinols and cannabinodiols such as cannabinodiol (CBND), cannabinodivarin (CBVD), cannabinol (CBN), cannabinol methylether (CBNM), cannabinol-C2 (CBN-C2), cannabinol-C4 (CBN-C4), cannabinolic acid (CBNA), cannabiorcool (CBN-C1), and cannabivarin (CBV); cannabitriols such as 10-ethoxy-9-hydroxy-delta-6a-tetrahydrocannabinol, 8,9-dihydroxy-delta-6a-tetrahydrocannabinol, cannabitriol (CBT), and cannabitriolvarin (CBTV); delta-8-tetrahydrocannabinols such as delta-8-tetrahydrocannabinol (48-THC) and delta-8-tetrahydrocannabinolic acid (48-THCA); delta-9-tetrahydrocannabinols such as delta-9-tetrahydrocannabinol (THC), delta-9-tetrahydrocannabinol-C4 (THC-C4), delta-9-tetrahydrocannabinolic acid A (THCA-A), delta-9-tetrahydrocannabinolic acid B (THCA-B), delta-9-tetrahydrocannabinolic acid-C4 (THCA-C4), delta-9-tetrahydrocannabiorcol (THC-C1), delta-9-tetrahydrocannabiorcolic acid (THCA-C1), delta-9-tetrahydrocannabivarin (THCV), and delta-9-tetrahydrocannabivarinic acid (THCVA); cannabichromanones such as cannabichromanone (CBCN-C5); cannabichromanone-C3 (CBCN-C3), and cannabicoumaronone (CBCON-C5); isocannabinoids such as (−)-delta-7-trans-(1R,3R,6R)-isotetrahydrocannabinol, (±)-delta-7-1,2-cis-(1R,3R,6S/1S,3 S,6R)-isotetrahydrocannabivarin, and (−)-delta-7-trans-(1R,3R,6R)-isotetrahydrocannabivarin; and other miscellaneous cannabinoids such as 10-oxo-delta-6a-tetrahydrocannabinol (OTHC), cannabichromanon (CBCF), cannabicitran, cannabifuran (CBF), cannabiglendol-C3, cannabiripsol (CBR), cannbicitran (CBT), dehydrocannabifuran (DCBF), delta-9-cis-tetrahydrocannabinol (cis-THC), trihydroxy-delta-9-tetrahydrocannabinol (triOH-THC), and 3,4,5,6-tetrahydro-7-hydroxy-alpha-alpha-2-trimethyl-9-n-propyl-2,6-methano-2H-1-benzoxocin-5-methanol (OH-iso-HHCV); and any stereoisomers, ethers, derivatives, metabolites, esters, salts, or carbon chain homologs thereof. In one embodiment, the at least one cannabinoid does not include any psychoactive cannabinoid. In one embodiment, the at least one cannabinoid does not include delta-9-tetrahydrocannabinol (THC). In one embodiment, the at least one cannabinoid comprises a synthetic cannabinoid.

In one embodiment, the beta-2 agonist is a short-acting beta agonist (SABA). Exemplary SABAs include, but are not limited to, albuterol (salbutamol), bitolterol, fenoterol, isoproterenol (isoprenaline), levalbuterol (levosalbutamol), mabuterol, metaproterenol (orciprenaline), naminterol, pirbuterol, procaterol, reproterol, ritodrine, salmefamol, and terbutaline. In one embodiment, the beta-2 agonist is a long-acting beta agonist (LABA). Exemplary LABAs include, but are not limited to, abediterol, arformoterol, AZD3199, bambuterol, B1-171800, carmoterol, clenbuterol, etanterol, flerbuterol, formoterol, GSK-159802, GSK-597901, GSK-678007, indacaterol, LAS100977, milveterol, olodaterol, PF-610355, pirbuterol, salmeterol, and vilanterol. Also considered are any stereoisomers, ethers, derivatives, metabolites, esters, salts, or carbon chain homologs of any beta-2 agonist.

The muscarinic antagonist can be any such compound known to those of skill in the art. In one embodiment, the muscarinic antagonist is an anticholinergic drug. Exemplary muscarinic antagonists include, but are not limited to, tiotropium, ipratropium, glycopyrronium, oxitropium, aclidinium, trospium, meclidinium, and salts thereof, including but not limited to bromide salts.

In one embodiment, the formulation further comprises at least one corticosteroid. Exemplary corticosteroids include, but are not limited to, hydrocortisone, beclomethasone, budesonide, fluticasone, mometasone, ciclesonide, flunisolide, and any stereoisomers, ethers, derivatives, metabolites, esters, salts, or carbon chain homologs thereof. In one embodiment, the formulation does not comprise a corticosteroid.

pMDI Formulations

In one aspect, the present invention relates to a formulation for inhalation via a pressurized metered dose inhaler (pMDI), wherein the pMDI formulation comprises a therapeutic composition comprising at least one cannabinoid and at least one additional therapeutic agent selected from the group consisting of a beta-2 agonist and a muscarinic antagonist. In some embodiments, the pMDI formulation optionally further comprises a solvent, a propellant, a co-solvent, a surfactant, and/or a stabilizer.

In one embodiment, the pMDI formulation comprises at least one solvent. In some embodiments, the solvent may aid in delivery of the formulation, such as for use in an inhaler device. In one embodiment, the formulation comprises at least one solvent selected from the group consisting water, buffered water, ethanol, propylene glycol, polyethylene glycol, erythritol, xylitol, mannitol, sorbitol, diethylene glycol monoethyl ether (Transcutol), and glycerol. In one embodiment, the formulation comprises a mixture of two or more solvents.

In one embodiment, the pMDI formulation further comprises at least one propellant. In various embodiments, a propellant comprises a pharmacologically inert liquid with a boiling point of from about room temperature (25° C.) to about −25° C. which exerts a high vapor pressure at room temperature. Without wishing to be bound by theory, inclusion of a propellant in a pharmaceutical composition of the present disclosure provides for pressurization of the composition in a canister assembly of the pMDI system and upon activation of the pMDI system, the high vapor pressure of the propellant in the pMDI system forces a metered amount of pharmaceutical composition out through the metering valve and the propellant very rapidly vaporizes, dispersing the pharmaceutical composition as an aerosol. In various embodiments, a propellant may be a hydrofluorocarbon, for example, a hydrofluoroalkane such as 1,1,1,2-tetrafluoroethane (HFA 134a), 1,1,1,2,3,3,3-heptafluoropropane (HFA 227ea), 1,1-difluoroethane (HFA-152a), difluoromethane (HFA-32), or a mixture thereof.

A pharmaceutical composition in accordance with various embodiments can comprise from about 50% to about 95% propellant.

In one embodiment, the pMDI formulation further comprises a surfactant. In some embodiments, the surfactant may aid in suspension of the formulation, such as in a solvent, or in delivery of the formulation. In some embodiments, the surfactant is present in an amount adequate for improving solubility. In some embodiments, the surfactant is present in an amount of up to 10% w/w. In some embodiments, additional or alternative surfactants can be applied if confirmed for the uses as provided herein. Exemplary surfactants include, but are not limited to, Span® 85, polyvinylpyrrolidone, oleic acid, sunflower oil, lecithin, phosphatidylcholine, isopropyl myristate, stearic acid, medium and long chain triglycerides (including Labrasol), polysorbate 20, polysorbate 80, or other ethoxylated surfactant, sorbitan trioleate, and other sorbitan surfactants.

In one embodiment, the pMDI formulation further comprises a stabilizer. In one embodiment, the stabilizer comprises an antioxidant. In one embodiment, the stabilizer comprises an antioxidant such as ascorbic acid. In one embodiment, the stabilizer comprises a chelating agent such as ethylenediaminetetraacetic acid (EDTA). In one embodiment, the stabilizer comprises an acid. Exemplary acids include, but are not limited to, carboxylic acids (mono, di or poly), halogen containing acids such as hydrochloric acid (HCl), hydrobromic acid (HBr), etc., sulfur containing acids such as sulfuric acid, sulfonic acids, sulfinyl acids, etc., phosphoric containing acids such as phosphoric acid, polyphosphoric acid, etc. or mixtures and combinations thereof. In one embodiment, the stabilizer comprises hydrochloric acid. In one embodiment, the stabilizer comprises a base. Exemplary bases include, but are not limited to, hydroxides, alkoxides, bicarbonates, phosphates and carbonates of alkali or alkaline earth metals. In one embodiment, the stabilizer comprises sodium hydroxide. In one embodiment, the stabilizer comprises potassium hydroxide.

In one embodiment, the stable aerosol inhalation composition comprises about 0.001% to about 25% w/w cannabinoid, about 0.001% to about 5% additional therapeutic(s), about 1% to about 50% ethanol, about 0.0001% to about 25% water, and at least one propellant.

In one embodiment, the stable aerosol inhalation composition comprises (a) about 0.001% w/w to about 7.5% w/w CBD, (b) about 0.001% w/w to about 7.5% w/w CBG, (c) about 0.001% w/w to about 7.5% w/w CBC, (d) about 0.001% w/w to about 0.5% w/w of Glycopyrronium Bromide, (e) about 0.001% w/w to about 0.5% w/w of Indacaterol Maleate, (f) about 1% w/w to about 25% w/w of ethanol as a co-solvent (g) about 0.0001% w/w to about 5% w/w of water and (h) HFA 134a propellant.

In one embodiment, the stable aerosol inhalation composition comprises (a) about 0.001% w/w to about 7.5% w/w CBD, (b) about 0.001% w/w to about 7.5% w/w CBG, (c) about 0.001% w/w to about 7.5% w/w CBN, (d) about 0.001% w/w to about 0.5% w/w of Glycopyrronium Bromide, (e) about 0.001% w/w to about 0.5% w/w of Indacaterol Maleate, (f) about 1% w/w to about 25% w/w of ethanol as a co-solvent (g) about 0.0001% w/w to about 5% w/w of water and (h) HFA 134a propellant.

In one embodiment, the stable aerosol inhalation composition comprises (a) about 0.001% w/w to about 7.5% w/w CBD, (b) about 0.001% w/w to about 7.5% w/w CBG, (c) about 0.001% w/w to about 7.5% w/w CBDV, (d) about 0.001% w/w to about 0.5% w/w of Glycopyrronium Bromide, (e) about 0.001% w/w to about 0.5% w/w of Indacaterol Maleate, (f) about 1% w/w to about 25% w/w of ethanol as a co-solvent (g) about 0.0001% w/w to about 5% w/w of water and (h) HFA 134a propellant.

In one embodiment, the stable aerosol inhalation composition comprises (a) about 0.001% w/w to about 7.5% w/w CBD, (b) about 0.001% w/w to about 7.5% w/w CBG, (c) about 0.001% w/w to about 7.5% w/w CBC, (d) about 0.001% w/w to about 7.5% w/w CBN, (e) about 0.001% w/w to about 7.5% w/w CBDV, (f) about 0.001% w/w to about 0.5% w/w of Glycopyrronium Bromide, (g) about 0.001% w/w to about 0.5% w/w of Indacaterol Maleate (h) about 1% w/w to about 25% w/w of ethanol as a co-solvent (i) about 0.0001% w/w to about 5% w/w of water and (h) HFA 134a propellant.

pMDI Devices

In one aspect, the present invention is directed to pressurized metered dose inhaler devices for the delivery of a pMDI formulation described herein. A pMDI device can comprise a delivery device consisting of a canister, a metering valve configured to sealably attach to a canister and to deliver a particular quantity of a composition contained within the canister (i.e., a metered dose) per actuation of the valve, and an actuator. A metering valve can be sealably attached to a canister to produce a canister assembly suitable to sealably contain a pressurized pharmaceutical composition. A pMDI device can also comprise a pharmaceutical composition contained within a sealed pMDI canister assembly.

In various embodiments, a pMDI system can comprise a plurality of pMDI canister assemblies or assembled pMDI devices (i.e., an assembled pMDI device including a filled canister assembly and an actuator), with each pMDI canister assembly or assembled pMDI device containing a different pharmaceutical formulation. Different pMDI canister assemblies or assembled pMDI devices containing different pharmaceutical formulations may be selected by a patient or a consumer in response to various factors, such as different desired therapeutic benefits, different sedative effects, different flavor profiles, and various combinations of the foregoing factors.

In various embodiments, an assembled pMDI device is configured to deliver the pharmaceutical composition in the form of droplets of a respirable size suitable for pulmonary administration. In various embodiments, a pMDI device, including the pharmaceutical composition, is configured to provide an aerosol particle size having a relatively uniform particle size distribution, for example, with substantially all, or at least about 90%, or at least about 80%, or at least about 70%, or at least about 60%, or at least about 50%, of the particles ranging between about 0.1 and about 25 microns, or between about 0.5 and about 10 microns, or between about 1.0 and about 5.0 microns. Particles larger than 25 microns may be deposited in the oropharyngeal cavity, while particles smaller than about 0.5 micron may fail to be deposited in the lungs and be lost due to exhalation. In various embodiments, the aerosol particle size produced by a pMDI device can be measured by cascade impaction and characterized by the mass median aerodynamic diameter (MMAD, i.e., the value for which 50% of the particles are larger or smaller). In various embodiments, the MMAD is between about 0.5 and about 10 microns, or between about 1.0 and about 5.0 microns.

In various embodiments, a pMDI device will comprise an actuator having an orifice with an orifice diameter. Preferably, the actuator orifice has a diameter in the range of from about 0.10 mm to about 0.70 mm, and more preferably in the range of from about 0.20 mm to about 0.70 mm. In various embodiments, the orifice diameter is in the range of from about 0.20 to about 0.60 mm.

In various embodiments, a metering valve used in a pMDI device can be configured to deliver a volume of a pharmaceutical composition in a range of from about 25 to about 100 microliters per actuation. In various embodiments, a pMDI device can be configured with a metering valve configured to deliver about a 50 microliter volume of a pharmaceutical composition.

In various embodiments, a pMDI device will comprise an actuator with an actuation or dose counter for counting the number of actuations of the system. The actuation or dose counter may be mechanical or electronic.

### Soft Mist and Nebulizer Formulations

In one aspect, the present invention relates to a formulation for inhalation via a nebulizer or soft mist inhaler, wherein the formulation comprises a therapeutic composition comprising at least one cannabinoid and at least one additional therapeutic agent selected from the group consisting of a beta-2 agonist and a muscarinic antagonist. In some embodiments, the formulation optionally further comprises water, a co-solvent, a surfactant, and/or a stabilizer.

In one embodiment, the formulation comprises water. In one embodiment, the formulation comprises at least one solvent selected from the group consisting of saline, buffered saline, water, buffered water, ethanol, propylene glycol, polyethylene glycol, sorbitol, and glycerol. In one embodiment, the formulation comprises a mixture of two or more solvents. In one embodiment, the formulation comprises normal saline (i.e., about 0.9% saline having about 9 g NaCl per liter of solution).

In one embodiment, the solution/emulsion/suspension formulation further comprises a surfactant. In some embodiments, the surfactant may aid in suspension of the formulation, such as in a solvent, or in delivery of the formulation. In some embodiments, the surfactant is present in an amount adequate for improving solubility. In some embodiments, the surfactant is present in an amount of up to 10% w/w. In some embodiments, additional or alternative surfactants can be applied if confirmed for the uses as provided herein. Exemplary surfactants include, but are not limited to, Span® 85, polyvinylpyrrolidone, oleic acid, sunflower oil, lecithin, phosphatidylcholine, isopropyl myristate, stearic acid, medium and long chain triglycerides (including Labrasol), polysorbate 20, polysorbate 80, or other ethoxylated surfactant, sorbitan trioleate, and other sorbitan surfactants.

In one embodiment, the formulation further comprises a stabilizer. In one embodiment, the stabilizer comprises an organic acid, an inorganic acid, an antioxidant or a buffer salt. In one embodiment, the stabilizer comprises an antioxidant and/or buffer salts. In one embodiment, the stabilizer comprises an antioxidant such as ascorbic acid. In one embodiment, the stabilizer comprises a chelating agent such as ethylenediaminetetraacetic acid (EDTA).

In one embodiment, the stable aerosol inhalation composition comprises about 0.001% to about 25% w/w cannabinoid, about 0.001% to about 5% additional therapeutic(s), about 1% to about 99% water, about 0.0001% to about 25% ethanol, and at least one stabilizer.

### Dry Powder Formulations

In one aspect, the present invention relates to dry powder formulations for inhalation, the dry powder formulation comprising at least one cannabinoid and at least one additional therapeutic agent selected from the group consisting of a beta-2 agonist and a muscarinic antagonist. In one embodiment, the dry powder formulation comprises a beta-2 agonist and a muscarinic antagonist. In one embodiment, the dry powder formulation further comprises an excipient.

In one embodiment, the dry power formulation is formulated as nucleus soft agglomerates, binary or ternary powder blends containing up to 99.9% total cannabinoids dependent on the beta-2 agonist and a muscarinic antagonist used in the formulation.

In one embodiment, the dry powder formulation includes dry particles, said particles comprising at least one cannabinoid and at least one additional therapeutic agent selected from the group consisting of a beta-2 agonist and a muscarinic antagonist. In one embodiment, the dry particles are sized substantially between about 1-10 microns, based on the median mass aerodynamic diameter (MMAD) of the particles. In one embodiment, the formulation includes dry particles sized substantially between about 1-7 microns. In one embodiment, the formulation includes dry particles sized substantially between about 2-5 microns. In one embodiment, the formulation includes dry particles sized substantially between about 2-3 microns. Accordingly, in some embodiments, the smallest particles within the dry particle size range are at least about 1 micron, at least about 1.1 microns, at least about 1.2 microns, at least about 1.3 microns, at least about 1.4 microns, at least about 1.5 microns, at least about 1.6 microns, at least about 1.7 microns, at least about 1.8 microns, at least about 1.9 microns, or at least about 2 microns.

In some embodiments, the largest particles within the dry particle size range are no greater than about 10 microns, no greater than about 7 microns, no greater than about 6 microns, no greater than about 5 microns, no greater than about 4.5 microns, no greater than about 4 microns, no greater than about 3.5 microns, or no greater than about 3 microns.

In certain embodiments, no more than about 10% of the dry particles are less than about 1 micron. In certain embodiments, no more than about 10% of the dry particles are less than about 2 microns. In other embodiments, at least 90% of the dry particles are less than about 10 microns. In other embodiments, at least 90% of the dry particles are less than about 7 microns. In other embodiments, at least 90% of the dry particles are less than about 5 microns. In one embodiment, no more than about 10% of the dry particles are less than about 1 micron and at least 90% of the dry particles are less than about 10 microns. In one embodiment, no more than about 10% of the dry particles are less than about 1 micron and at least 90% of the dry particles are less than about 7 microns. In one embodiment, no more than about 10% of the dry particles are less than about 2 microns and at least 90% of the dry particles are less than about 5 microns. In one embodiment, no more than about 10% of the dry particles are less than about 2 microns and at least 90% of the dry particles are less than about 3 microns.

As would be understood by a person skilled in the art, the particle size ranges described herein are not absolute ranges. For example, a dry particle mixture of the present invention with a size range of about 2-5 microns can contain a portion of particles that are smaller or larger than the about 2-5 microns range. In one embodiment, the particle size value as presented for any particular component of the formulations of the present invention represents a D90 value, wherein 90% of the particles sizes of the mixture are less than the D90 value. In another embodiment, the particle size range represents a particles size distribution (PSD) wherein a percentage of the particles of the mixture lie within the listed range. For example, a dry particle size range of about 2-5 microns can represent a mixture of dry particles having at least 50% of the particles in the range of about 2-5 microns, but more preferably a higher percentage, such as, but not limited to: 60%, 70%, 80%, 90%, 95%, 97%, 98% or even 99%.

It should be appreciated that the component particles may be spherical or of any other shape desired. In one embodiment the particles may have an uneven or a “dimpled” surface. In such embodiments, the uneven surface may increase the ability of additional components to cling to the dry particles and produce a uniform coating. For example, the additional component may be a therapeutic such as menthol assuring that every dry particle that hits a cough receptor is coated with menthol which will suppress the cough reflex. The uneven surface may also produce a relative turbulence as the particles travel through the air, thus providing the particles with aerodynamic lift. In such embodiments, particles having such shape may be more readily entrained, and to remain entrained, in the air inhaled by a subject, thereby improving the ability of the component particles to travel and to be retained in the alveoli and airways of the subject.

In one embodiment, the dry powder formulation includes an amino acid. In one embodiment, the amino acid is leucine. In one embodiment, leucine acts as a stabilizer, by reducing by any degree the degradation of a composition of the invention. In another embodiment, leucine prevents the degradation of a composition of the invention by acting as a buffer by virtue of its buffering capabilities. In another embodiment, leucine acts as a powder flow enhancer. In another embodiment, the leucine in a composition of the invention improves the flow of the powder. In another embodiment, the leucine in a composition of the invention causes the particles of the powder formulation to be more readily entrained, and to remain entrained, in the air inhaled by a subject, thereby improving the ability of the composition particles to travel to and to be retained in the alveoli and airways. In one embodiment, the percentage of leucine in the formulation is between 0.5% and 10%. In some embodiments, the percentage of leucine in the formulation is between 1.5% and 2.5%. In other embodiments, the percentage of leucine in the formulation is between 0.5% and 2.5%. In yet other embodiments the percentage of leucine in the formulation is between 1.5% and 5%. In one embodiment, the percentage of leucine in the formulation is about 2.5%. In another embodiment, the percentage of leucine in the formulation is about 5%. In another embodiment, the percentage of leucine in the formulation is about 7.5%. In another embodiment, the percentage of leucine in the formulation is about 10%.

In one embodiment, the dry powder formulation further comprises excipients. As contemplated herein, one embodiment of an excipient is a bulking agent. Bulking agents may include inhalable sugars that are generally solid at room temperature. The sugar can be milled into a particulate formulation, either by itself, or co-milled with the at least one cannabinoid and/or the additional therapeutic component. Without limitation, examples of suitable sugars are lactose, sucrose, raffinose, trehalose, fructose, dextrose, glucose, maltose, lecithin, mannitol, or combinations thereof. In one embodiment, the sugar is lactose. In another embodiment, the lactose is coarse lactose. In another embodiment, the sugar is alpha monohydrate lactose. The sugar may be a natural or a synthetic sugar, and may include any analogs or derivatives of sugars. It should be appreciated that any form of sugar approved as an excipient may be used as a carrier in the production of the therapeutic component. While not required, the sugar is preferably of a pharmaceutical grade as would be understood by those skilled in the art. Preferably, the pharmaceutical grade sugar used to be milled by itself, co-milled with a therapeutic component or to create the flowable mixture is a non-spheronized sugar. The pharmaceutical grade sugar may be prepared in a non-spheronized form prior to dry or wet admixture with the therapeutic component. For example, the pharmaceutical grade sugar may be first prepared in a non-spheronized form by freeze drying, milling, micronizing or the like. In certain embodiments, the pharmaceutical grade sugar may be subjected to milling, bashing, grinding, crushing, cutting, sieving or other physical degradation process as understood by those skilled in the art, which ultimately reduces the particle size of the sugar and results in a non-spheronized sugar.

It should be appreciated that there are no limitations to the ratio of therapeutic component to sugar used, and the actual ratio used will be based on the concentration of therapeutic component desired in the component particles. Accordingly, in one embodiment the concentration of sugar is at least about 50%. In another embodiment, the concentration of sugar is between about 50% and about 99%.

Some materials that may useful in the formulation of the present invention include pharmaceutically acceptable ternary excipients such as metal stearates, amino acids such as leucine, L-leucine, D-leucine, DL-leucine, isoleucine, isoleucine, trileucine, lysine, valine, arginine, aspartic acid, threonine, methionine, phenylalanine, alginic acid, alginate salts, derivatives of amino acids, for example aspartame or acesulfame K; cellulose derivatives, phospholipids. In one embodiment, each material is “acceptable” in the sense of being compatible with the other ingredients of the formulation, including the at least one cannabinoid and/or the additional therapeutic component, and not injurious to the subject.

In one embodiment, the dry powder formulation comprises a cough suppressant component. In one embodiment, the additional cough suppressant component is menthol. In one embodiment, the concentration of menthol in the formulation is between about 0.5% and about 20%. As contemplated herein, any form of menthol, such as a solid form of menthol can be used for processing into menthol particles, powder, solution or suspension useful within the present invention. Non-limiting examples of solid forms of menthol include powders, crystals, solidified distillate, flakes, and pressed articles. In one embodiment, menthol is in the form of crystals. Menthol can be processed into particles of a size ranging from about 5 microns (μm) to about 10 μm using any method known in the art. In another embodiment, menthol is admixed with a sugar, such as lactose. In some embodiments of the wet process, the menthol is processed in a liquid carrier. In another embodiment, the additional cough suppressant component is mint. In one embodiment, the concentration of mint in the formulation is between about 0.5% and about 20%. As contemplated herein, any form of mint, such as a solid form of mint can be used for processing into mint particles, powder, solution or suspension useful within the present invention.

In one embodiment, the dry powder formulation comprises a cough suppressant component having particles sized substantially between 5 and 10 microns. In another embodiment, the additional cough suppressant component may include benzocaine. It should be appreciated that the additional cough suppressant component can include any compound approved for suppressing cough. By selectively including menthol particles between 5-10 microns, these non-respirable menthol particles can reduce cough in the subject's upper airways. Accordingly, in some embodiments, the smallest particles within the additional cough suppressant component particle size range are at least about 5 microns, at least about 6 microns, at least about 7 microns, or at least about 8 microns. In some embodiments, the largest particles within the additional cough suppressant component particle size range are no greater than about 10 microns, no greater than about 9 microns, no greater than about 8 microns, or no greater than about 7 microns. In certain embodiments, no more than about 10% of the additional cough suppressant particles are less than about 5 microns. In other embodiments, at least 90% of the additional cough suppressant particles are less than about 10 microns. In other embodiments, at least 90% of the additional cough suppressant particles are less than about 8 microns. In one embodiment, no more than about 10% of the additional cough suppressant particles are less than 4 microns and at least 90% of the additional cough suppressant particles are less than about 10 microns. In one embodiment, no more than about 10% of the additional cough suppressant particles are less than about 5 microns and at least 90% of the additional cough suppressant particles are less than about 8 microns. Although in the preferred embodiment the additional cough suppressant component is composed of particles substantially in the range of 5-10 microns, the additional cough suppressant component can comprise particles in a broader range. In one embodiment, the additional cough suppressant component can comprise particles in the range of 5-25 microns. In another embodiment, the additional cough suppressant component comprises particles substantially in the range of 5-50 microns. In yet another embodiment, the additional cough suppressant component comprises particles substantially in the range of 5-100 microns.

In one embodiment, the dry powder formulation comprises a cough suppressant component having particles sized substantially between 10-200 microns. In one embodiment, this large particle size cough suppressant component is added to the formulation instead of, or in addition to, the cough suppressant component in the range of 5-10 previously discussed. Accordingly, the formulation of the present invention can comprise two cough suppressant components, wherein each cough suppressant component has a substantially different particle size distribution. The 10-200 micron cough suppressant component may reduce a cough caused by irritation of the oro-pharynx, the glottis vocal cords and other anatomic regions more proximal or closer to the mouth that contain receptors that can trigger cough or trigger other unwanted sensations. As contemplated herein, these larger particles are substantially prohibited from entering the sub-glottic airways. Accordingly, in some embodiments, the smallest particles within the cough suppressant component particle size range are at least about 10 micron, at least about 12 micron, at least about 20 micron, at least about 30 micron, or at least about 50 micron. In some embodiments, the largest particles within the cough suppressant component particle size range are no greater than about 200 micron, no greater than about 150 micron, no greater than about 120 micron, no greater than about 100 micron, no greater than about 90 micron, or no greater than about 80 micron. In certain embodiments, no more than about 10% of the cough suppressant component particles are less than about 10 micron. In certain embodiments, no more than about 10% of the cough suppressant component particles are less than about 20 micron.

In some embodiments, at least 90% of the cough suppressant component particles are less than about 200 micron. In other embodiments, at least 90% of the cough suppressant component particles are less than about 150 micron. In other embodiments, at least 90% of the cough suppressant component particles are less than about 100 micron. In one embodiment, no more than about 10% of the cough suppressant component particles are less than 10 micron and at least 90% of the cough suppressant component particles are less than about 200 micron. In one embodiment, no more than about 10% of the cough suppressant component particles are less than about 12 micron and at least 90% of the cough suppressant component particles are less than about 100 micron. In one embodiment, the cough suppressant component includes menthol particles between about 10-200 microns in size. In another embodiment, the cough suppressant component having particles between about 10-200 microns in size may include benzocaine. It should be appreciated that the cough suppressant component having particles between about 10-200 microns in size can include any compound approved for suppressing cough. In another example, the addition of at least one component in the formulation of the present invention other than the therapeutic component may act to dilute the therapeutic-containing particles and decrease cough caused by irritation in the oro-pharynx, vocal cords and other anatomic regions proximal to the trachea.

In one embodiment, the dry powder formulation comprises a flavor component having particles sized substantially between about 10-1000 microns. In one embodiment, the flavor component is composed of particles substantially in the range of about 10-200 micron. In a preferred embodiment, the flavor component is composed of particles substantially in the range of about 10-100 micron. This flavor component utilizes such embedded larger particles that may impact the subject in the oral cavity to produce a desired flavor. Further, by limiting such flavor component particles to larger than about 10 microns in size, these particles are limited in their ability to enter into the subject's lungs. Accordingly, in some embodiments, the smallest particles within the flavoring component particle size range are at least about 10 micron, at least about 12 micron, at least about 20 micron, at least about 30 micron, or at least about 50 micron. In some embodiments, the largest particles within the flavoring component particle size range are no greater than about 1000 micron, no greater than about 500 micron, no greater than about 200 micron, no greater than about 150 micron, no greater than about 120 micron, no greater than about 100 micron, no greater than about 90 micron, or no greater than about 80 micron. In certain embodiments, no more than about 10% of the flavor component particles are less than about 10 micron. In certain embodiments, no more than about 10% of the flavor component particles are less than about 20 micron. In other embodiments, at least 90% of the flavor component particles are less than about 1000 micron. In other embodiments, at least 90% of the flavor component particles are less than about 500 micron. In other embodiments, at least 90% of the flavor component particles are less than about 200 micron. In other embodiments, at least 90% of the flavor component particles are less than about 150 micron. In other embodiments, at least 90% of the flavor component particles are less than about 100 micron. In one embodiment, no more than about 10% of the flavor component particles are less than 10 micron and at least 90% of the flavor component particles are less than about 1000 micron. In one embodiment, no more than about 10% of the flavor component particles are less than 10 micron and at least 90% of the flavor component particles are less than about 200 micron. In one embodiment, no more than about 10% of the flavor component particles are less than about 10 micron and at least 90% of the flavor component particles are less than about 100 micron. In one embodiment, the flavor component is mint. In another embodiment, the flavor component is menthol. In other embodiments, the flavor component may include tobacco, fruit flavors, or food grade flavorings used in candy or baking. It should be appreciated that the flavor compound may be any flavoring compound known in the art, preferably a regulatory-approved flavoring compound.

In various embodiments, the relative weight percentage of each component in the dry powder formulation of the present invention can be varied to achieve different characteristics. In one embodiment, the dry powder formulation can be about 1-20% by weight flavor component. In one embodiment, the dry powder formulation comprises 1-5% by weight flavor component. In one embodiment, the dry powder formulation comprises about 1% to about 10% by weight cough suppressant. In one embodiment, the dry powder formulation comprises about 0.5% to about 5% by weight cough suppressant. In various embodiments, the remaining portion of the formulation, aside from any flavor components, cough suppressant components, carriers, or other components, is the therapeutic compounds component.

In one embodiment, the dry powder formulation comprises lactose. In one embodiment, the percentage of lactose in the dry powder formulation is between 50% and 99%. In one embodiment, the percentage of lactose in the dry powder formulation is between 50% and 80%. In some embodiments, the percentage of lactose in the dry powder formulation is between 75% and 90%. In other embodiments, the percentage of lactose in the dry powder formulation is between 75% and 85%. In yet other embodiments the percentage of lactose in the dry powder formulation is between 80% and 90%. In yet other embodiments the percentage of lactose in the dry powder formulation is between 80% and 99%. In one embodiment, the percentage of lactose in the dry powder formulation is about 50%. In one embodiment, the percentage of lactose in the dry powder formulation is about 60%. In one embodiment, the percentage of lactose in the dry powder formulation is about 70%. In one embodiment, the percentage of lactose in the dry powder formulation is about 80%. In another embodiment, the percentage of lactose in the dry powder formulation is about 90%. In another embodiment, the percentage of lactose in the dry powder formulation is about 95%. In another embodiment, the percentage of lactose in the dry powder formulation is about 99%.

In one embodiment, the percentage of menthol in the dry powder formulation is between 0% and 20%. In some embodiments, the percentage of menthol in the dry powder formulation is between 5% and 20%. In other embodiments, the percentage of menthol in the dry powder formulation is between 5% and 15%. In yet other embodiments the percentage of menthol in the dry powder formulation is between 10% and 20%. In one embodiment, the percentage of menthol in the dry powder formulation is about 5%. In another embodiment, the percentage of menthol in the dry powder formulation is about 20%.

In one embodiment, the percentage of mint in the dry powder formulation is between 0% and 20%. In some embodiments, the percentage of mint in the dry powder formulation is between 5% and 20%. In other embodiments, the percentage of mint in the dry powder formulation is between 5% and 15%. In yet other embodiments the percentage of mint in the dry powder formulation is between 10% and 20%. In one embodiment, the percentage of mint in the dry powder formulation is about 5%. In another embodiment, the percentage of mint in the dry powder formulation is about 20%.

### Methods of Use

In one aspect, the invention relates to methods for controlling the amount of at least one cannabinoid and the amount of additional therapeutic selected from the group consisting of beta-2 agonist and muscarinic antagonist delivered by the pMDI formulation or the dry powder formulation, the method including increasing, decreasing, or maintaining the amount of cannabinoid and the amount of additional therapeutic in the formulation inhaled by a subject. For example, an exemplary method includes the steps of identifying a concentration of at least one cannabinoid and the amount of additional therapeutic for a subject to inhale, identifying the total dose of at least one cannabinoid for a subject to inhale, identifying a concentration of the additional therapeutic for a subject to inhale, identifying the total dose of the additional therapeutic for a subject to inhale, and providing a subject with an amount of a pMDI formulation or a dry powder formulation comprising the at least one cannabinoid and the amount of additional therapeutic having the identified concentration of the at least one cannabinoid and comprising additional therapeutic having the identified concentration, such that the total amount of the at least one cannabinoid and additional therapeutic in the formulation equals the total dose of the at least one cannabinoid and the total dose of additional therapeutic.

It should be appreciated that any manner of increasing, decreasing or maintaining the total dose of the at least one cannabinoid in a formulation can be combined with any manner of increasing, decreasing or maintaining the amount of the additional therapeutic in the formulation.

As contemplated herein, there is no limitation to the particular formulation amount of pMDI solution/suspension or dry powder, or the concentration of the at least one cannabinoid within the total formulation amount, but rather, the present invention relates to the ability to alter one or both of these parameters when delivering a total dose of the at least one cannabinoid and the additional therapeutic selected from the group consisting of a beta-2 agonist and a muscarinic antagonist to a subject via a pMDI inhaler or a dry powder inhaler. Further, there is no limitation to the actual amount of formulation inhaled per inhalation. Such amounts can be dependent on the functionality of the inhaler used, or it can be user performance dependent, where a user elects to take a shallower, or deeper, inhalation through the inhaler used. Furthermore, by administering the total therapeutic dose across multiple inhalations, the subject can more consistently insure uptake of the total therapeutic dose, as any user error occurring during a single inhalation is ultimately corrected through one or more subsequent inhalations.

### Methods of Manufacture of Dry Powder Formulations

The present invention also relates to methods of making the dry powder formulations of the present invention. In one embodiment, the methods comprise dry mixing. In one embodiment, the methods comprise wet mixing.

In on embodiment, an exemplary dry process of producing any one of the dry powder formulations described herein comprises the steps of milling a solid component, mixing the dry component with at least one excipient such as lactose, and optionally adding additional components as desired. In some embodiments, the at least one cannabinoid and/or the additional therapeutic is not bound to any other components of the dry powder formulation. In one embodiment, the dry powder formulation contains distinct particles of at least one cannabinoid, distinct particles of the additional therapeutic, and/or distinct particles of other components of the dry powder formulation, such as a sugar. In one embodiment, the at least one cannabinoid, the additional therapeutic, and/or any additional component dry particles are at least partially bound and/or agglomerated. size (e.g., 10-1000 micron) for the flavor component being added.

Any method of blending particles in and for the methods and dry powder formulations of the present invention is contemplated here. The blending can be conducted in one or more steps, in a continuous, batch, or semi-batch process. For example, if two or more excipients are used, they can be blended together before, or at the same time as, being blended with the pharmaceutical agent microparticles.

The blending can be carried out using essentially any technique or device suitable for combining the microparticles with one or more other materials (e.g., excipients) effective to achieve uniformity of blend. The blending process may be performed using a variety of blenders. Representative examples of suitable blenders include V-blenders, slant-cone blenders, cube blenders, bin blenders, static continuous blenders, dynamic continuous blenders, orbital screw blenders, planetary blenders, Forberg blenders, horizontal double-arm blenders, horizontal high intensity mixers, vertical high intensity mixers, stirring vane mixers, twin cone mixers, drum mixers, and tumble blenders. The blender preferably is of a strict sanitary design required for pharmaceutical products.

Tumble blenders are often preferred for batch operation. In one embodiment, blending is accomplished by aseptically combining two or more components in a suitable container. One example of a tumble blender is the TURBULA™, distributed by Glen Mills Inc., Clifton, N.J., USA, and made by Willy A. Bachofen AG, Maschinenfabrik, Basel, Switzerland.

For continuous or semi-continuous operation, the blender optionally may be provided with a rotary feeder, screw conveyor, or other feeder mechanism for controlled introduction of one or more of the dry powder components into the blender.

A milling step is used to fracture and/or deagglomerate the blended particles, to achieve a desired particle size and size distribution, as well as to enhance distribution of the particles within the blend. Any method of milling can be used to form the particles of the invention, as understood by one of ordinary skill in the art. A variety of milling processes and equipment known in the art may be used. Examples include hammer mills, ball mills, roller mills, disc grinders, jet milling and the like. Preferably, a dry milling process is used.

In one embodiment, the dry powder formulation is produced using a wet process. In one embodiment, the at least one cannabinoid and the additional therapeutic selected from the group consisting of a beta-2 agonist and a muscarinic antagonist are admixed with excipients. As contemplated herein, any liquid carrier may be used in the process of producing the solution or suspension. In one embodiment, the liquid carrier is water. Preferably, the liquid carrier is one in which the components of the formulation are either soluble or suspendable. Accordingly, the liquid carrier may be any liquid or liquids with which the components of the formulation, either alone or in combination, form a flowable mixture or suspension which is preferably of a generally uniform composition.

In one embodiment, the mixture is then atomized and then dried, such as via a spray drier. Alternatively, the process may optionally be performed via fluid bed drying, wherein the at least one cannabinoid and/or the additional therapeutic can instead be spray dried onto an excipient mixture. In one embodiment, the resulting particles are filtered, such as with a sieve, to remove any particles larger than a threshold size value. In one embodiment, the resulting particles are filtered again to remove any particles smaller than a threshold size value, resulting in the final dry powder formulation. In one embodiment, only one filtering step is needed. In one embodiment, two or more filtering steps are needed. In one embodiment, a flavor component may be added to the final formulation. In one embodiment, any number of processing steps may be performed to obtain the desired particle size (e.g., 10-1000 micron) for the flavor component being added.

The flowable mixtures are dried, such as via a spray drier, to produce composite particles of the flowable mixtures that are suitable for delivery to the alveoli and lower airways of a subject. It should be appreciated that there is no limitation to the method of drying the flowable mixtures. While a preferred method utilizes a spray drier, other drying techniques capable of producing appropriately sized particles may be used, such as fluidized bed drying. In one embodiment, the mixture is finely divided via passage through an orifice upon on entry to a spray dryer. In another embodiment, the flowable mixture may be passed through an atomizer, such as a rotary atomizer, to feed the flowable liquid into a spray dryer. Further still, any rate of drying may be used (e.g., slow or rapid rate drying), provided such rate of drying results in the formation of dry particles of the desired size range. Prior to the segregation of the desired particle size of the therapeutic component, the resultant particles formed via the spray drier may have a particle size from about 0.1 to about 5 micron.

Additional segregation/filtering of selected particle sizes may be performed both in the dry and the wet process. In the wet process, the operating conditions of the spray dryer may be adjusted so to produce particles which are sized so as to be able to travel to the alveoli and smaller airways of the lungs. For example, a rotary atomizer may be operated at a liquid feed rate from about 2 to about 20 ml/min, or from 2 to about 10 ml/min, or from about 2 to about 5 ml/min. Further, the rotary atomizer may be operated from about 10,000 to about 30,000 rpm, from about 15,000 to about 25,000 rpm, or from about 20,000 to about 25,000 rpm. It should be appreciated that particles of various sizes may be obtained by spray drying, and particles having the desired particle size may be more specifically selected when filtered, such as via one or more sieving steps, as described elsewhere herein. The spray dryer may be operated at temperatures sufficiently high to cause the liquid carrier to rapidly evolve without raising the temperature of the sugar and the at least one cannabinoid and/or the additional therapeutic component within the mixture to a point at which these compounds begin to degrade. Accordingly, the spray dryer may be operated with an inlet temperature from about 120° C. to about 170° C., and an outlet temperature from about 70° C. to about 100° C.

It should be appreciated that there is no limitation to the method of drying the flowable mixtures. Examples of methods for drying the flowable mixtures include, but are not limited to, spray drying, vacuum drying, and freeze drying. Further still, any rate of drying may be used (e.g., slow or rapid rate drying), provided such rate of drying results in the formation of dry particles of the desired size range.

As mentioned previously, in the wet process the liquid carrier is dried, such as via a fluidized bed dryer, to produce composite particles coated with menthol that are suitable for delivery to the alveoli and lower airways of a subject. It should be appreciated that there is no limitation to the method of drying the flowable mixture. While a preferred method utilizes a fluidized bed dryer, other drying techniques capable of removing the liquid carrier and leaving a uniform menthol coating on the dry particles may be used.

As contemplated herein, the particles of the present invention can be produced in relatively narrow size ranges via the use of at least one sieving step. In such an embodiment, the sieving step includes using a sieve corresponding to the minimum or maximum of the desired particle size range to eliminate particles from the mixture that are smaller or bigger than the desired range. For example, to obtain dry particles in the range of about 1-5 microns, a mixture of dry particles produced using the milling process described herein can be provided. The mixture of dry particles will have a size distribution that is dependent on the milling conditions used and/or the characteristics of the input mixture to the mill. The mixture of dry particles can first be passed through a 5 micron sieve, wherein substantially all of the particles smaller than 5 microns pass through the sieve and are collected. The particles passing through the sieve can then transferred to a 1 micron sieve, wherein substantially all of the particles greater than 1 micron do not pass through the sieve. The particles greater than 1 micron can be collected from the sieve, wherein the collected particles will be substantially sized in the range of 1-5 microns. Accordingly, such a process can be used to narrow the range of any mixture of particles to any of the desired particle size ranges as described herein throughout

In another embodiment, a mixture of dry particles can be provided that substantially meets either the minimum or maximum criteria of the desired particle size range. For example, if a dry particle size range of about 2-5 microns is desired, a mixture of dry particles can be provided wherein substantially all of the particles are less than 5 microns. Such a mixture can be produced by modifying the milling conditions, or when the particles are spray dried, by milling the spray dried material to result in a mixture of particles that are generally less than 5 microns. The mixture can then be transferred through a 2 micron sieve, wherein the particles not passing through the sieve are collected, and wherein the collected particles are substantially within the desired 2-3 micron range.

It is contemplated that the percentage of dry particles falling within the desired particle size range for any of the components of the formulation of the present invention can be dependent on the technique used to produce that component. For example, if the targeted size of the therapeutic component is in the range of 2-5 micron, it is understood that greater than 90% of that component will fall within the desired range when using a spray drying production technique on a relatively small scale. However, using a relatively large scale milling production technique may only yield greater than 70% of the therapeutic component within such a targeted range.

### Methods of Treatment

In one aspect, the present invention relates to methods of treating obstructive or inflammatory airways diseases, particularly asthma and COPD, in a subject in need thereof. In one embodiment, the method comprises the step of administering to the subject via inhalation a formulation, pMDI formation, or dry powder formulation discussed herein.

Other obstructive or inflammatory airways diseases and conditions to which the present invention is applicable include acute/adult respiratory distress syndrome (ARDS), chronic obstructive pulmonary or airways disease (COPD or COAD), including chronic bronchitis, or dyspnea associated therewith, emphysema, as well as exacerbation of airways hyperreactivity consequent to other drug therapy, in particular other inhaled drug therapy. The invention is also applicable to the treatment of bronchitis of whatever type or genesis including, e.g., acute, arachidic, catarrhal, croupus, chronic or phthinoid bronchitis. Further obstructive or inflammatory airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

### Therapeutic Formulations

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled pharmacologist can design and perform such modification with merely ordinary, if any, experimentation. Subjects to which administration of the pharmaceutical compositions of the disclosure is contemplated include, but are not limited to, humans and other primates, mammals including commercially relevant mammals such as non-human primates, cattle, pigs, horses, sheep, cats, and dogs.

Pharmaceutical compositions that are useful in the methods of the disclosure may be prepared, packaged, or sold in formulations suitable for ophthalmic, oral, rectal, vaginal, parenteral, topical, pulmonary, intranasal, buccal, intravenous, intracerebroventricular, intradermal, intramuscular, or another route of administration. Other contemplated formulations include projected nanoparticles, liposomal preparations, resealed erythrocytes containing the active ingredient, and immunogenic-based formulations.

A pharmaceutical composition of the disclosure may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the disclosure may further comprise one or more additional pharmaceutically active agents.

Controlled- or sustained-release formulations of a pharmaceutical composition of the disclosure may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intradermal, intrasternal injection, intratumoral, intravenous, intracerebroventricular and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the disclosure may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the disclosure are known in the art and described, for example in Remington's Pharmaceutical Sciences, 1985, Genaro, ed., Mack Publishing Co., Easton, PA), which is incorporated herein by reference.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1: Combining Cannabinoids with Muscarinic Antagonists and β-2 Agonists

An optimized HPLC assay method was developed for the assay of prototype formulations. The optimized HPLC assay conditions are presented in Table 1.

TABLE 1
Optimized HPLC assay method

| Mobile phase | A: Methanol:Water (20:80) with 1% Acetic acid and 0.1M SLS | | |
|---|---|---|---|
| | B: Acetonitrile:Methanol:Water (75:5:20) with 1% Acetic acid | | |
| | Gradient: | | |
| | Time | A (%) | B (%) |
| | 0.00 | 45 | 55 |
| | 5.00 | 95 | 5 |
| | 5.01 | 95 | 5 |
| | 8.50 | 95 | 5 |
| | 8.51 | 45 | 55 |
| | 10.00 | 45 | 55 |

| Column | Agilent Eclipse XDB-C18, 150 × 4.6 mm, 3.5 μm | |
|---|---|---|
| Flow rate | 1 | ml/min |
| Temperature | 35° | C. |
| Injection volume | 50 | μL |
| Runtime | 25 | minutes |
| Diluent | 70% Aqueous MeOH | |
| Detection | 230 | nm |

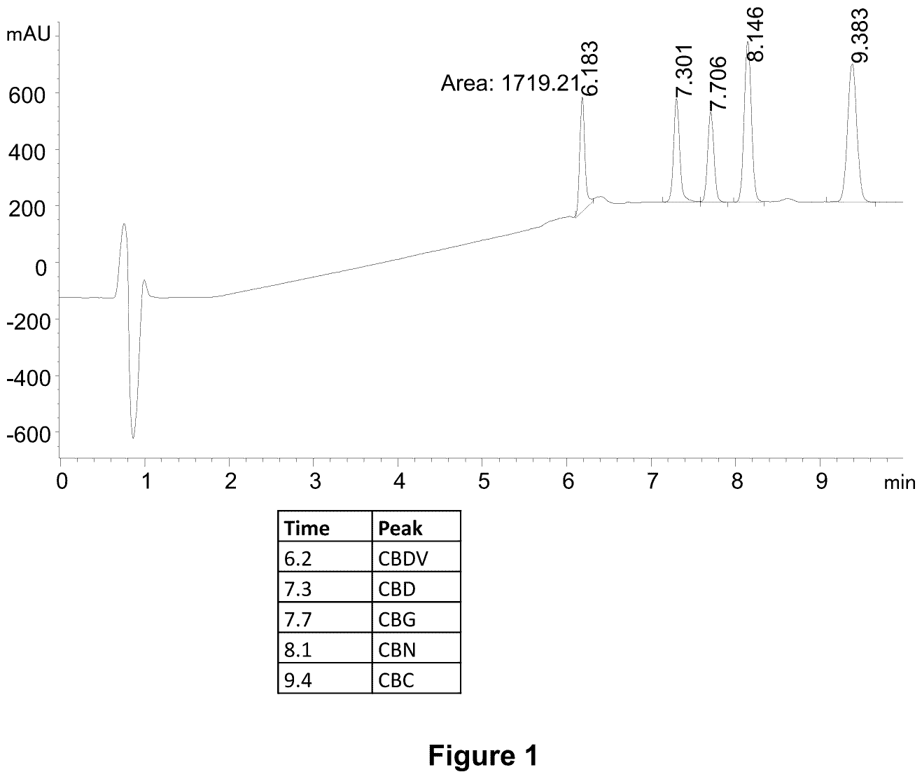

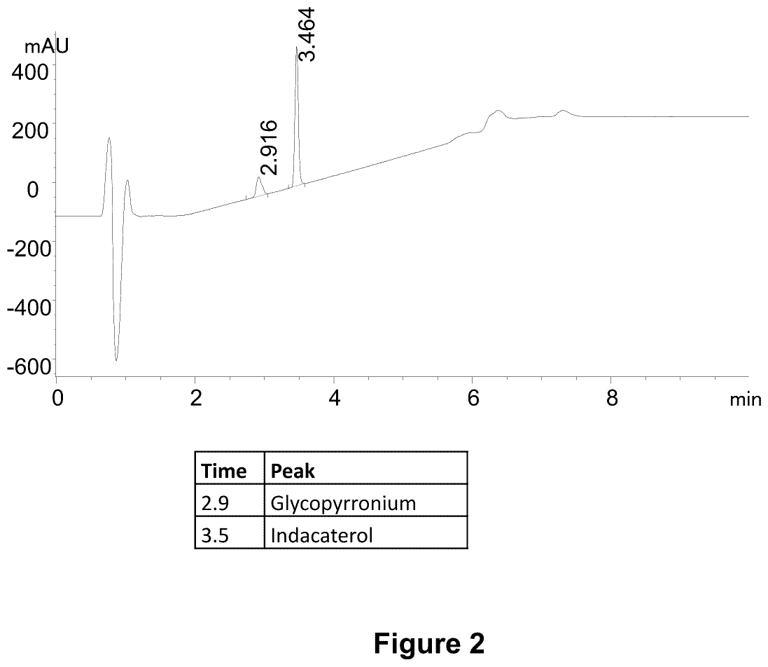

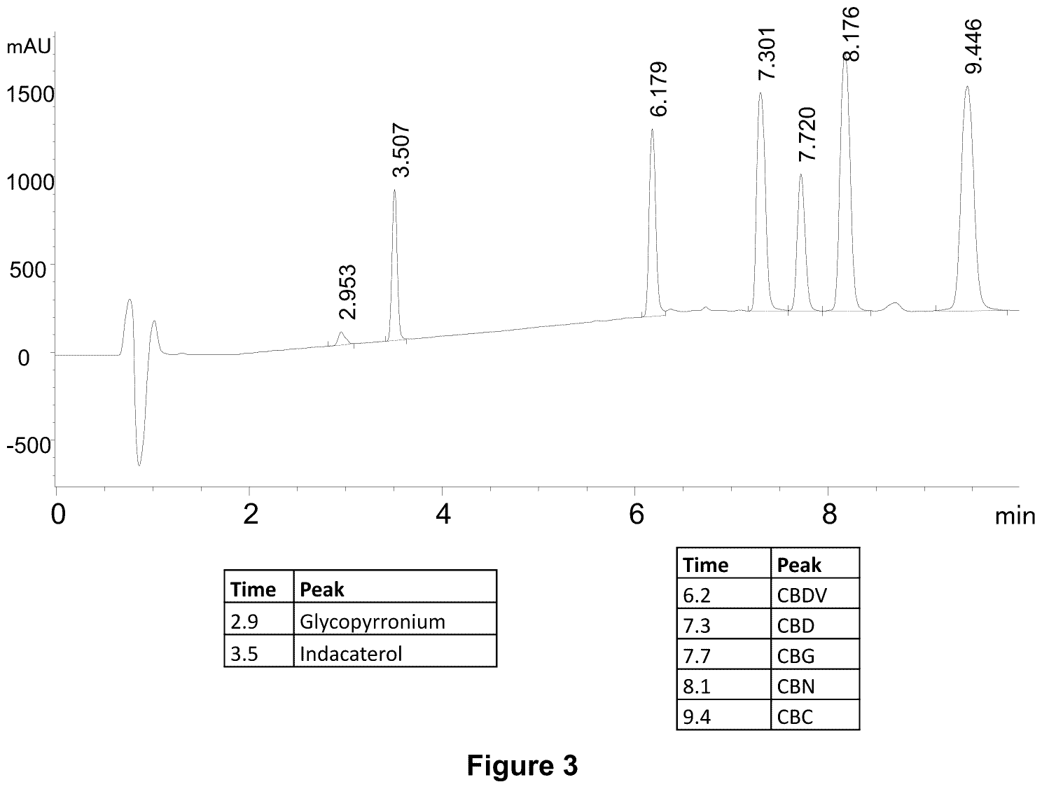

HPLC chromatogram of cannabinoids cannabichromevarin (CBDV), cannabidiol (CBD), cannabigerol (CBG), cannabinol (CBN), and cannabichromene (CBC) using the optimized assay are presented in FIG. 1. HPLC chromatogram of beta-2 agonist indacaterol and muscarinic antagonist glycopyrronium are presented in FIG. 2. An HPLC chromatogram of a mixture of a cannabinoids, indacaterol, and glycopyrronium is presented in FIG. 3.

The HPLC assay was used to develop chromatography standards. The results of the standardization assay is presented in Table 2.

TABLE 2
Typical standard reproducibility

| | Peak Area | | | | | | |
|---|---|---|---|---|---|---|---|
| Inj. | Glycopyrronium | Indacaterol | CBD | CBG | CBDV | CBN | CBC |
| 1 | 354.2 | 1646.9 | 1640.5 | 1648.5 | 1663.4 | 3407.8 | 3744.8 |
| 2 | 355.6 | 1647.2 | 1657.3 | 1666.3 | 1674.2 | 3417.8 | 3752.4 |
| 3 | 354.5 | 1645.1 | 1620.9 | 1664.1 | 1650.5 | 3390.1 | 3782.1 |
| 4 | 351.5 | 1645.9 | 1704.2 | 1648.1 | 1609.5 | 3383.6 | 3741.4 |
| 5 | 358.0 | 1671.3 | 1699.9 | 1678.5 | 1638.9 | 3417.5 | 3781.5 |
| Mean | 354.8 | 1651.3 | 1664.6 | 1661.1 | 1647.3 | 3403.4 | 3760.4 |
| STDEV | 2.4 | 11.2 | 36.6 | 12.9 | 25.0 | 15.8 | 19.9 |
| % RSD | 0.7 | 0.7 | 2.2 | 0.8 | 1.5 | 0.5 | 0.5 |

A series of prototype formulations were developed for testing with a next generation impactor (NGI). The prototype formulations are described in Table 3.

TABLE 3
Prototype formulations

| | Content | | | |
|---|---|---|---|---|
| | mg per Canister | | | |
| Formulation | 1 | 2 | 3 | 4 |
| CBD | 200 | 200 | 200 | 200 |
| CBG | 200 | 200 | 200 | 100 |
| CBC | 200 | 0 | 0 | 100 |
| CBN | 0 | 200 | 0 | 100 |
| CBV | 0 | 0 | 200 | 100 |
| Indacaterol Maleate | 25 | 25 | 25 | 25 |
| Glycopyronium Bromide | 12.5 | 12.5 | 12.5 | 12.5 |
| Ethanol* | 2160 | 2160 | 2160 | 2160 |
| Water** | 300 | 300 | 300 | 300 |
| HFA134a | 6902.5 | 6902.5 | 6902.5 | 6902.5 |
| Total | 10000 | 10000 | 10000 | 10000 |

*2.7 ml
**0.3 ml or as required

The total emitted dose from APIs ED 1 to ED 8 was then analyzed using the HPLC Assay. The results are shown in Table 4.

TABLE 4
Typical emitted dose - Formulation 1-4

| | Glyco- | | | | | | |
|---|---|---|---|---|---|---|---|
| API | pyrronium | Indacaterol | CBD | CBG | CBDV | CBN | CBC |
| ED 1 | 27.6 | 52.6 | 287.0 | | | | |
| ED 2 | 28.7 | 49.3 | 289.2 | | | | |
| ED 3 | 27.5 | 50.7 | 308.0 | 338.2 | | | |
| ED 4 | 28.5 | 52.2 | 316.2 | 353.3 | | | |
| ED 5 | 30.3 | 50.2 | 284.9 | 323.7 | | | |
| ED 6 | 32.4 | 47.4 | 292.6 | 332.9 | | | |
| ED 7 | 27.0 | 50.4 | 301.6 | 331.0 | 265.4 | 255.7 | 262.4 |
| ED 8 | 30.2 | 51.6 | 304.3 | 332.1 | 278.5 | 263.5 | 269.8 |
| Mean | 29.0 | 50.5 | 298.0 | 335.2 | 272.0 | 259.6 | 266.1 |
| STDEV | 1.8 | 1.7 | 11.2 | 10.0 | 9.3 | 5.5 | 5.2 |
| % RSD | 6 | 3 | 4 | 3 | 3 | 2 | 2 |

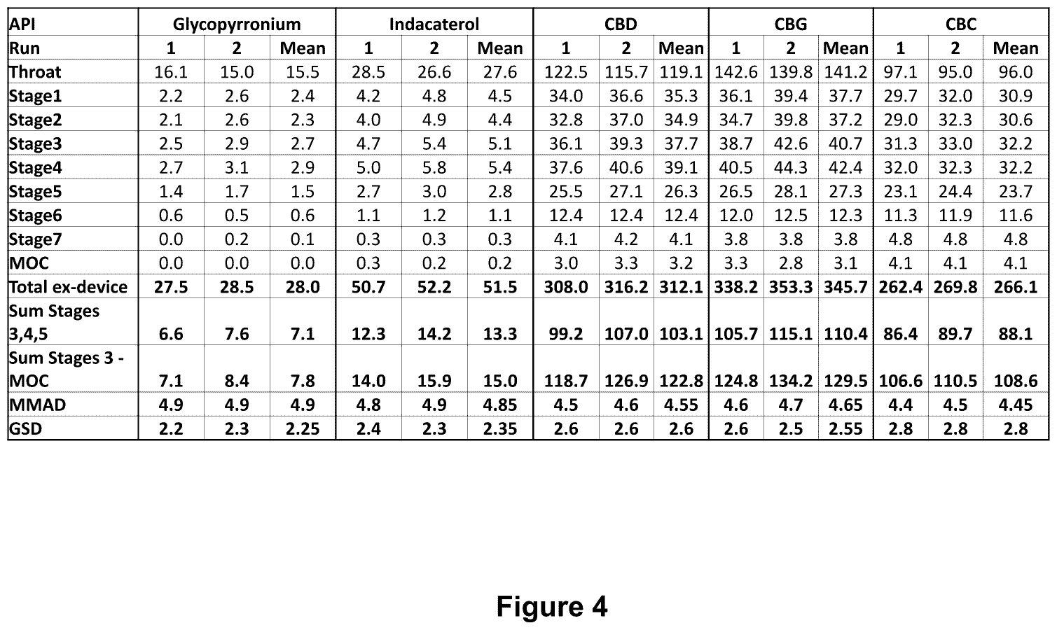

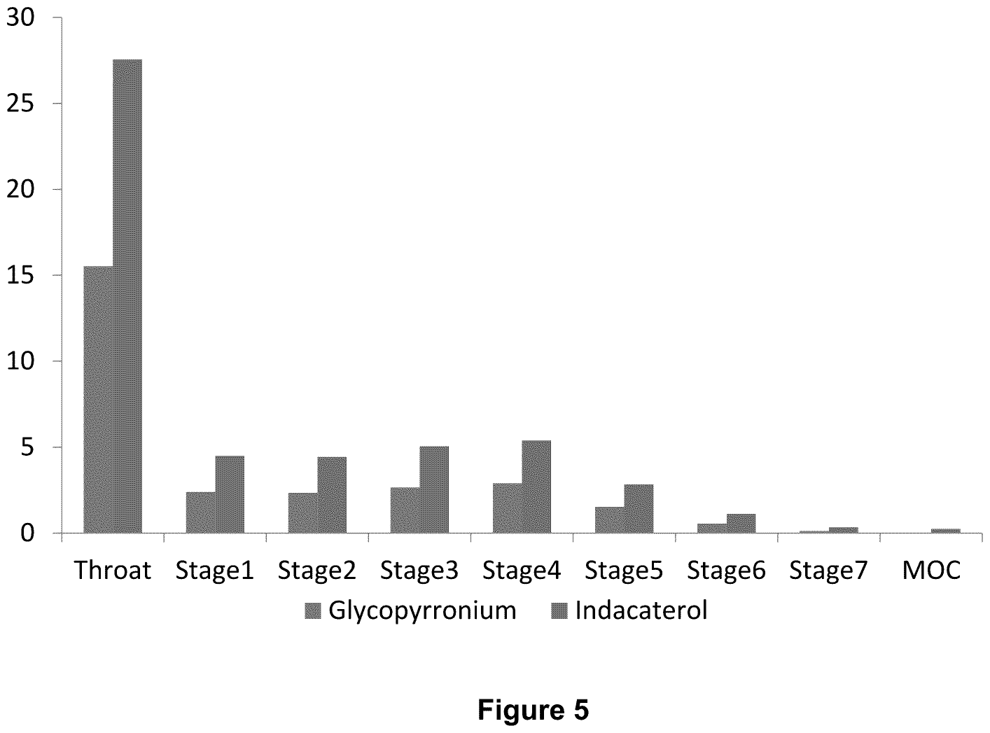

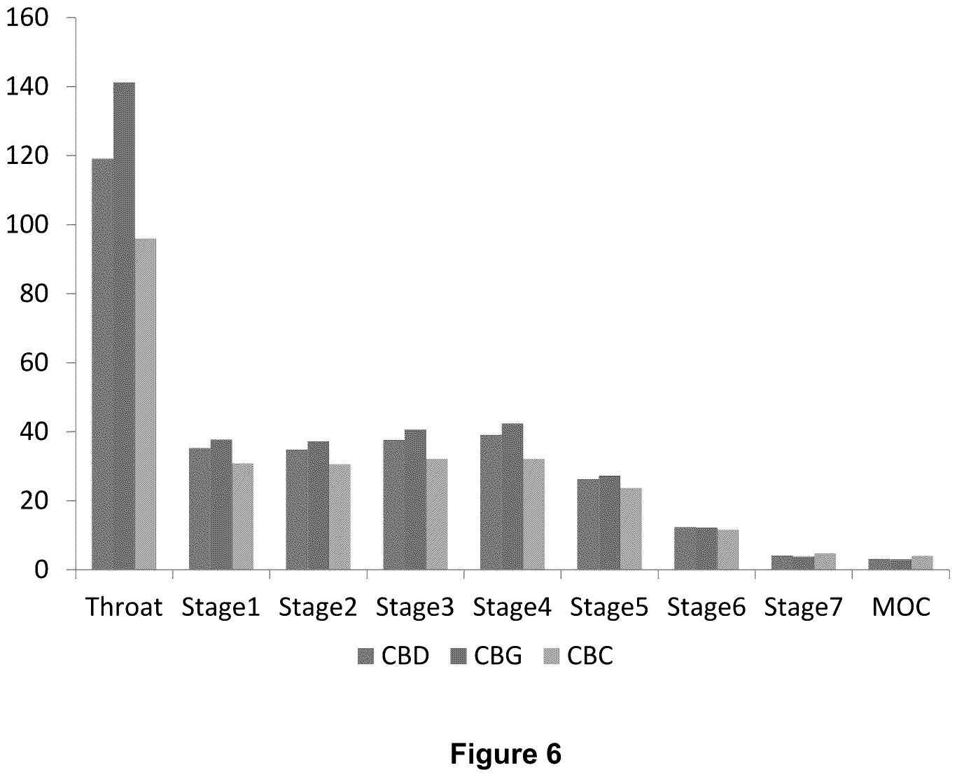

Formulation 1 was subjected to NGI testing. The distribution of the formulation components is tabulated in FIG. 4. The distribution of glycopyrronium and indacaterol are plotted in FIG. 5. The distribution of cannabinoids are plotted in FIG. 6. A summary of Formulation 1 NGI testing is presented below in Table 5.

The Next Generation Pharmaceutical Impactor (NGI) testing is performed using Copley and MSP instruments. Emitted dose or Ex-device is calculated as the total dose deposited in the NGI apparatus starting from Throat stage to Micro-orifice collector (MOC). The Fine Particle Dose (FPD) is calculated as the total dose deposited in Stages 3 to MOC of the NGI apparatus. Mass Median Aerodynamic Diameter (MMAD) and Geometric Standard Deviation (GSD) is calculated from aerodynamic particle size distribution of particles in different stages of the NGI apparatus.

TABLE 5
Formulation 1 NGI testing

| API | Glycopyrronium | Indacaterol | CBD | CBG | CBC |
|---|---|---|---|---|---|
| Target Emitted | 27.0 | 42.0 | 300.0 | 300.0 | 300.0 |
| dose (μg) | | | | | |
| Actual Emitted | 28.0 | 51.5 | 312.1 | 345.7 | 266.1 |
| dose (μg) | | | | | |
| % Emitted dose | 104 | 123 | 104 | 115 | 89 |
| Fine Particle | 7.8 | 15.0 | 122.8 | 129.5 | 108.6 |
| Dose (μg) | | | | | |
| % Fine | 29 | 36 | 41 | 43 | 36 |
| Particle Dose | | | | | |
| MMAD | 4.9 | 4.9 | 4.6 | 4.7 | 4.5 |
| GSD | 2 | 2 | 3 | 3 | 3 |

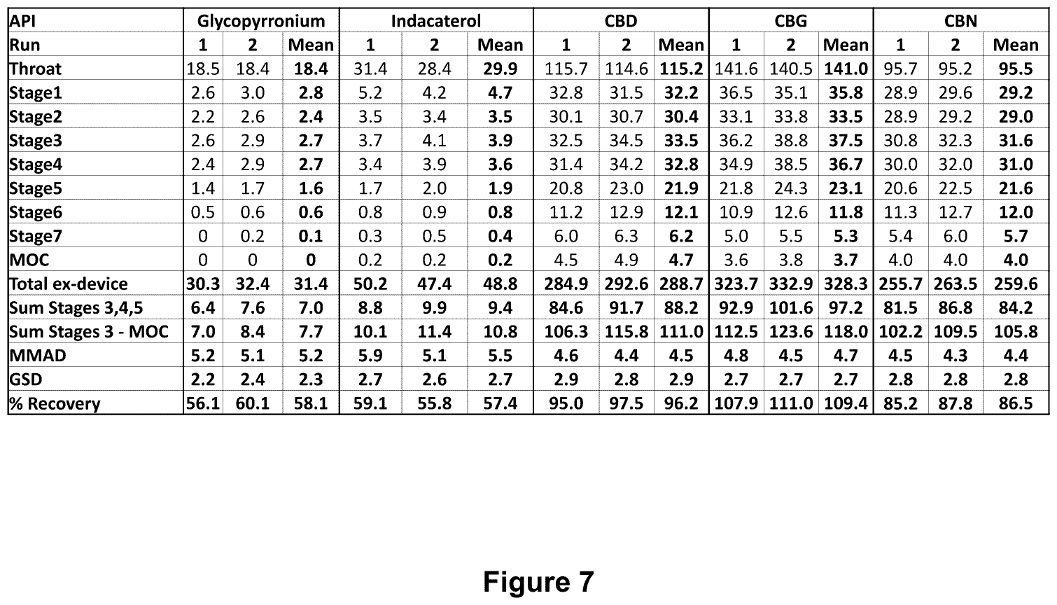

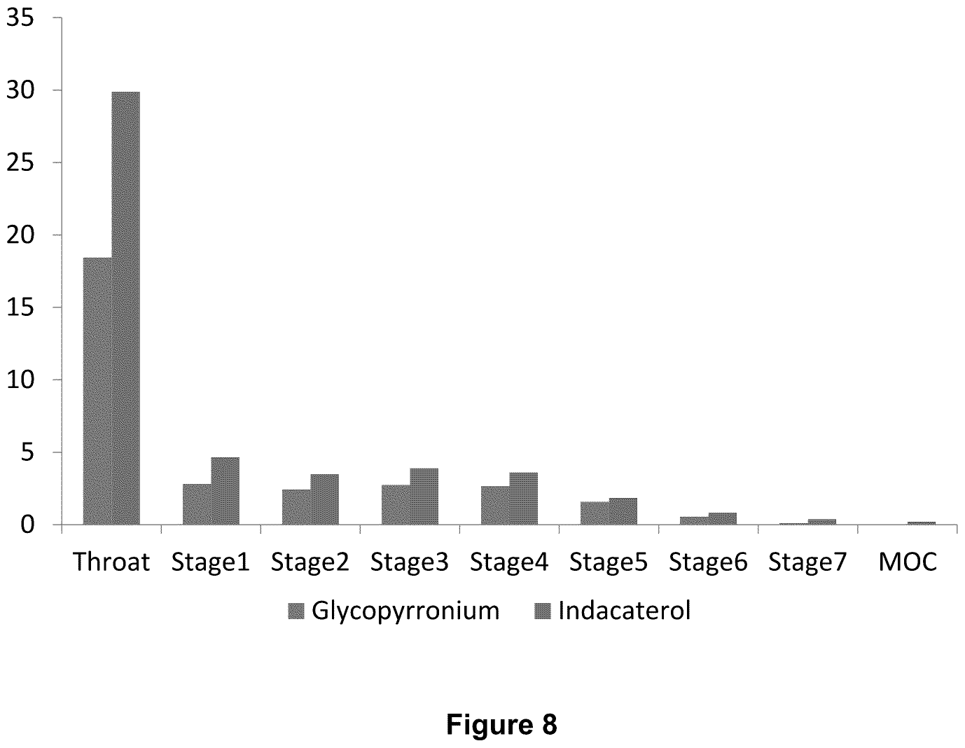

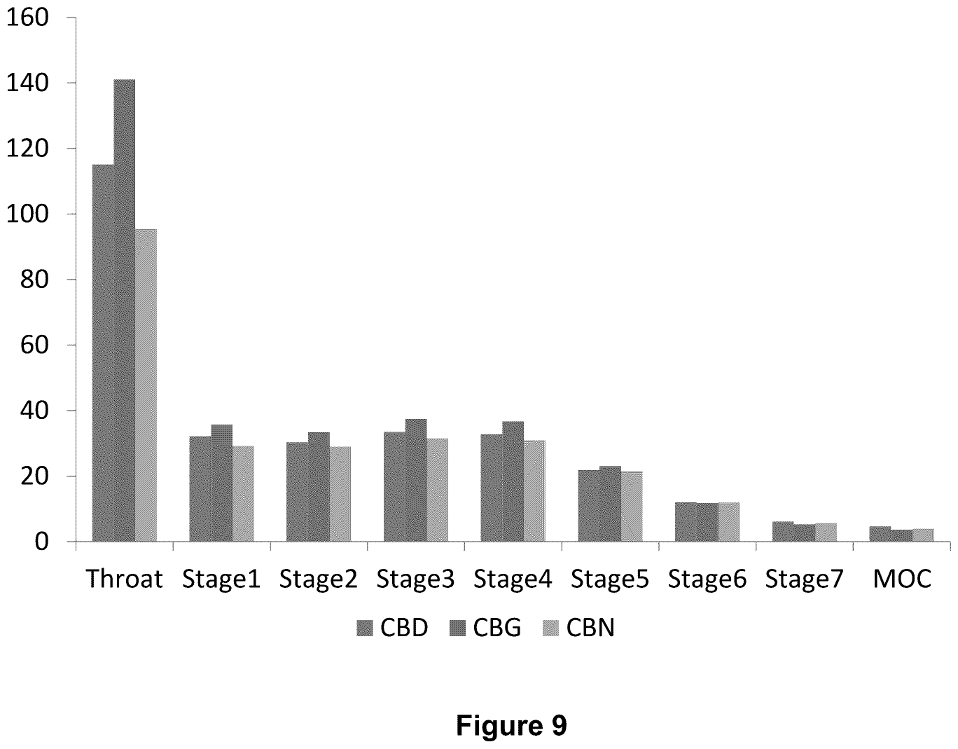

Formulation 2 was subjected to NGI testing. The distribution of the formulation components is tabulated in FIG. 7. The distribution of glycopyrronium and indacaterol are plotted in FIG. 8. The distribution of cannabinoids are plotted in FIG. 9. A summary of Formulation 1 NGI testing is presented below in Table 6.

TABLE 6
Formulation 2 NGI testing

| API | Glycopyrronium | Indacaterol | CBD | CBG | CBN |
|---|---|---|---|---|---|
| Target Emitted | 27 | 42 | 300 | 300 | 300 |
| dose (μg) | | | | | |
| Actual Emitted | 31.4 | 48.8 | 288.7 | 328.3 | 259.6 |
| dose (μg) | | | | | |
| % Emitted dose | 116 | 116 | 96 | 109 | 87 |
| Fine Particle | 7.7 | 10.8 | 111.0 | 118.0 | 105.8 |
| Dose (μg) | | | | | |
| % Fine | 28 | 26 | 37 | 39 | 35 |
| Particle Dose | | | | | |
| MMAD | 5.15 | 5.5 | 4.5 | 4.65 | 4.4 |
| GSD | 2 | 3 | 3 | 3 | 3 |

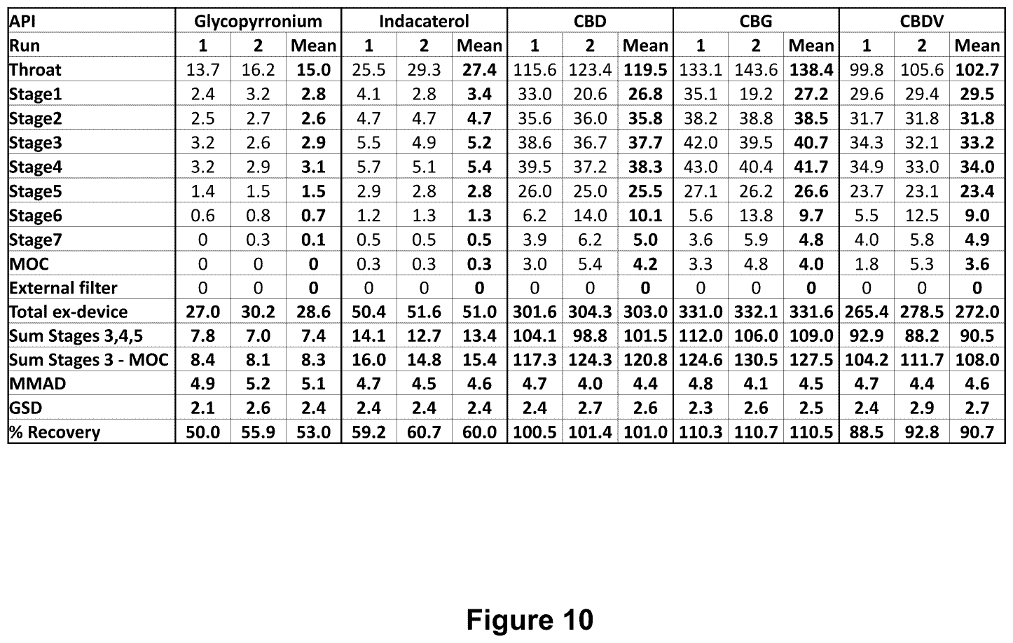

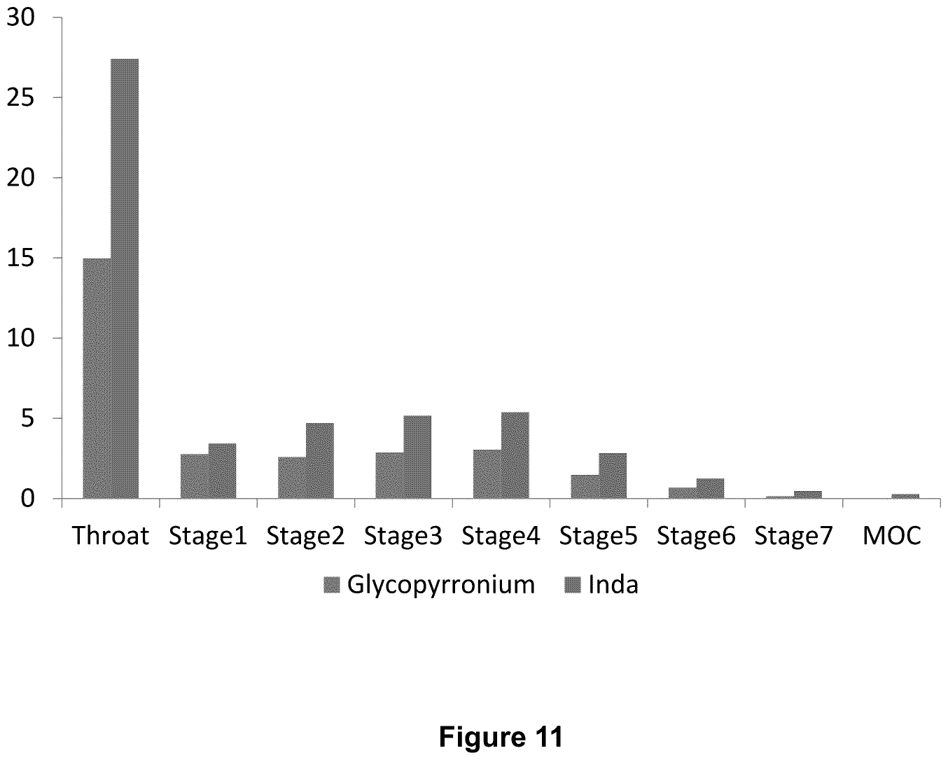

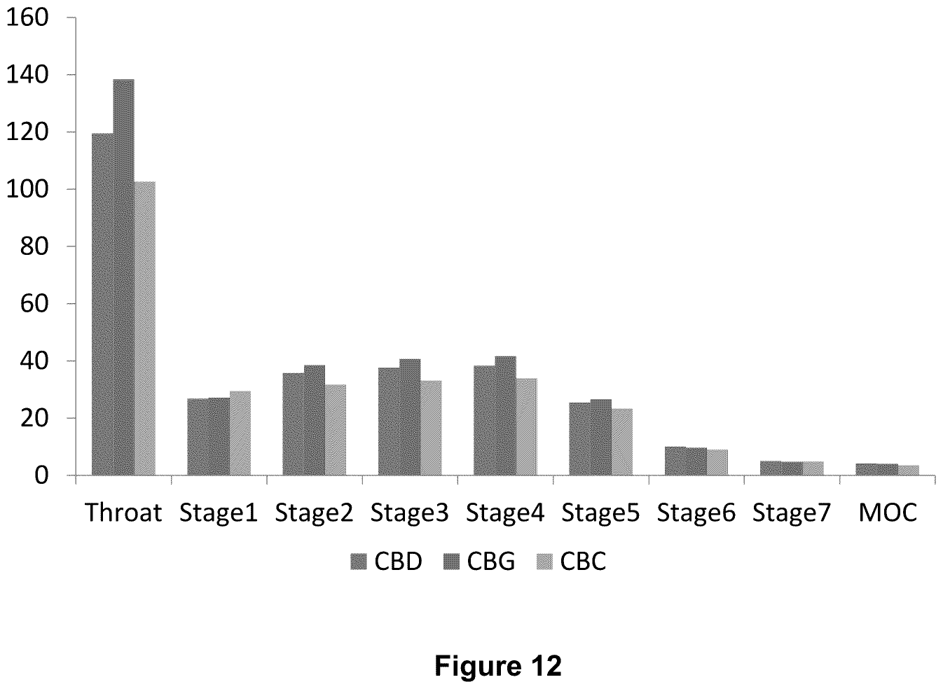

Formulation 3 was subjected to NGI testing. The distribution of the formulation components is tabulated in FIG. 10. The distribution of glycopyrronium and indacaterol are plotted in FIG. 11. The distribution of cannabinoids are plotted in FIG. 12. A summary of Formulation 1 NGI testing is presented below in Table 7.

TABLE 7
Formulation 3 NGI testing

| API | Glycopyrronium | Indacaterol | CBD | CBG | CBDV |
|---|---|---|---|---|---|
| Target Emitted | 27 | 42 | 300 | 300 | 300 |
| dose (μg) | | | | | |
| Actual Emitted | 28.6 | 51.0 | 303.0 | 331.6 | 272.0 |
| dose (μg) | | | | | |
| % Emitted dose | 106 | 121 | 101 | 111 | 91 |
| Fine Particle | 8.3 | 15.4 | 120.8 | 127.5 | 108.0 |
| Dose (μg) | | | | | |
| % Fine | 31 | 37 | 40 | 43 | 36 |
| Particle Dose | | | | | |
| MMAD | 5.1 | 4.6 | 4.4 | 4.5 | 4.6 |
| GSD | 2 | 3 | 3 | 3 | 3 |

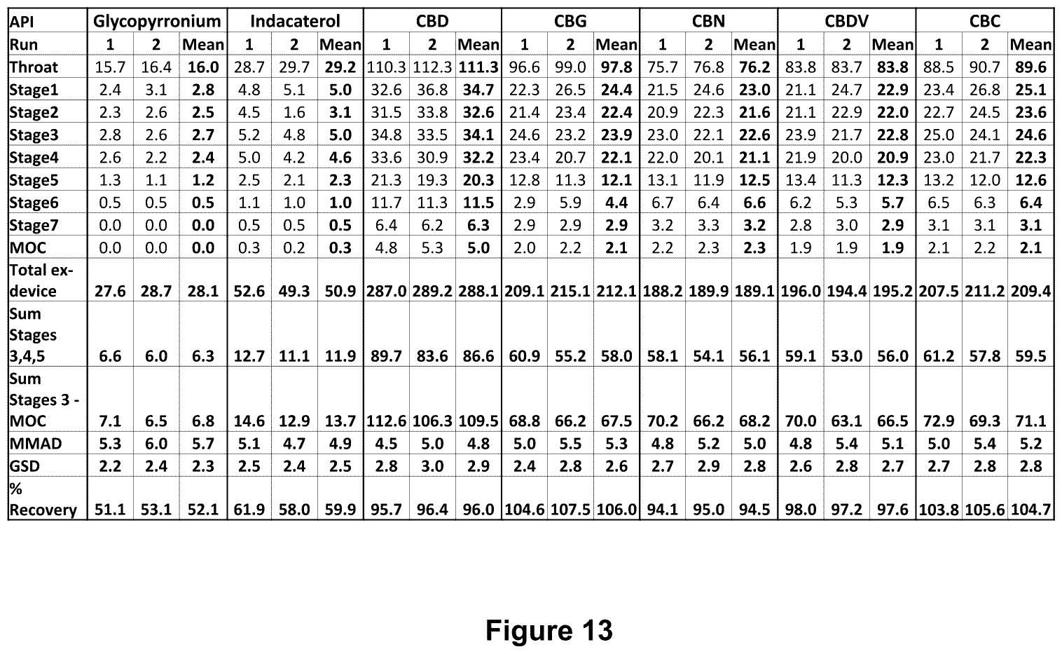

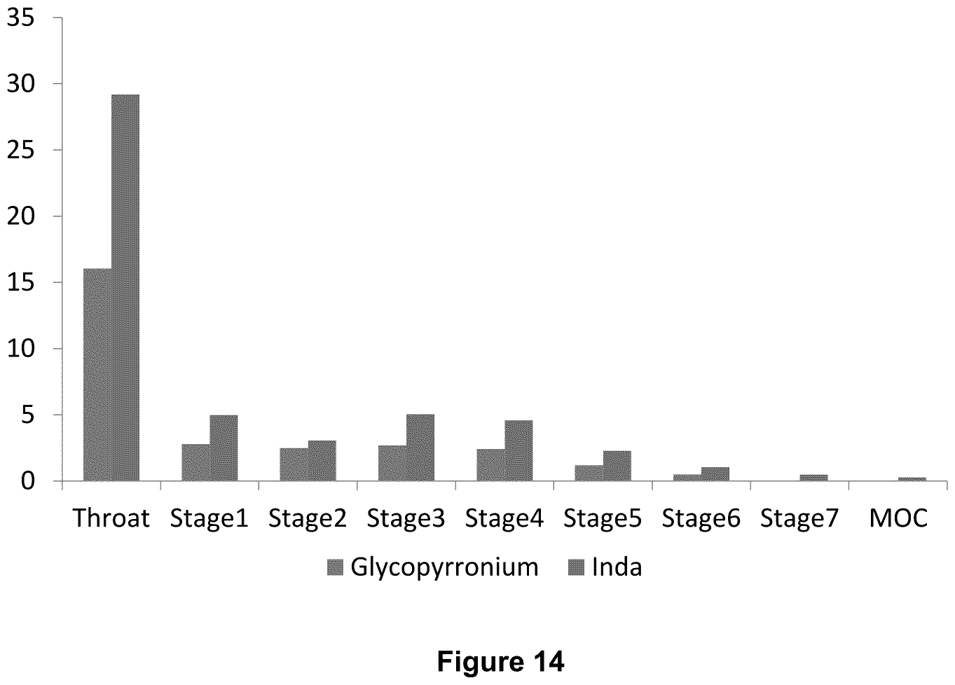

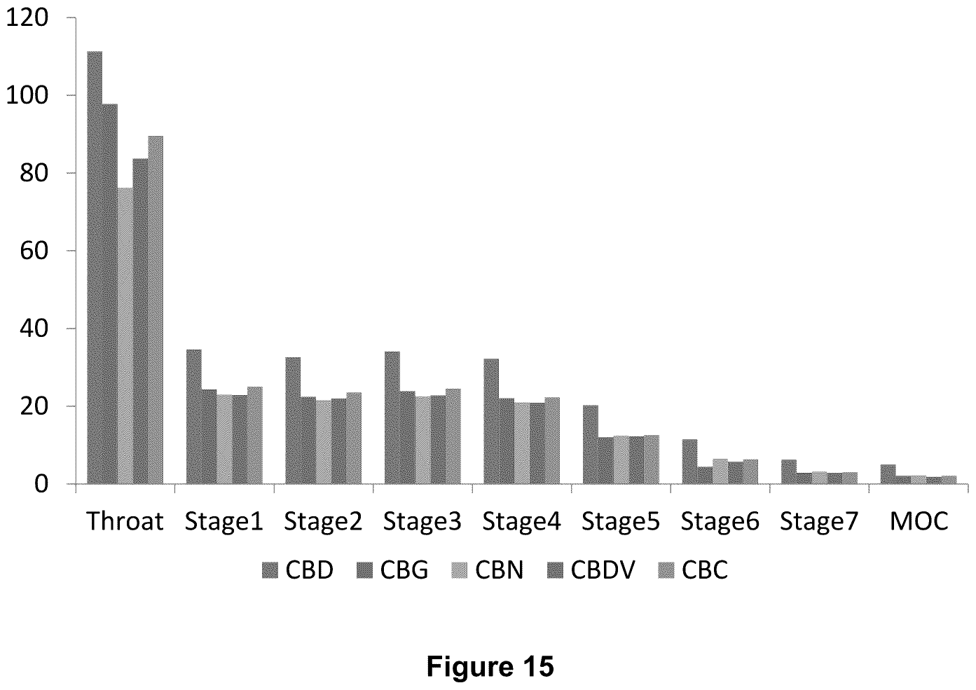

Formulation 4 was subjected to NGI testing. The distribution of the formulation components is tabulated in FIG. 13. The distribution of glycopyrronium and indacaterol are plotted in FIG. 14. The distribution of cannabinoids are plotted in FIG. 15. A summary of Formulation 1 NGI testing is presented below in Table 8.

TABLE 8
Formulation 4 NGI testing

| API | Glycopyrronium | Indacaterol | CBD | CBG | CBN | CBDV | CBC |
|---|---|---|---|---|---|---|---|
| Target | 27 | 42 | 300 | 200 | 200 | 200 | 200 |
| Emitted | | | | | | | |
| dose (μg) | | | | | | | |
| Actual | 28.1 | 50.9 | 288.1 | 212.1 | 189.1 | 195.2 | 209.4 |
| Emitted | | | | | | | |
| dose (μg) | | | | | | | |
| % | 104 | 121 | 96 | 106 | 95 | 98 | 105 |
| Emitted | | | | | | | |
| dose | | | | | | | |
| Fine | 6.8 | 13.7 | 109.5 | 67.5 | 68.2 | 66.5 | 71.1 |
| Particle | | | | | | | |
| Dose | | | | | | | |
| (μg) | | | | | | | |
| % Fine | 25 | 33 | 36 | 34 | 34 | 33 | 36 |
| Particle | | | | | | | |
| Dose | | | | | | | |
| MMAD | 5.65 | 4.9 | 4.75 | 5.25 | 5 | 5.1 | 5.2 |
| GSD | 2 | 2 | 3 | 3 | 3 | 3 | 3 |

The pMDI formulation may contain a cannabinoid or a combination or cannabinoids; a muscarinic antagonist such as Glycopyrronium, Tiotropium, Umeclidinium, Aclidinium etc.; and a beta-2 agonist such as Indacaterol, Salmeterol, Formoterol, Olodaterol, Salbutamol etc.

In addition to cannabinoids, beta-2 agonist, and muscarinic antagonist, the formulation may also include a co-solvent such as ethanol, water, buffered water, propylene glycol, Polyethylene glycol and glycerol; a propellant such as HFA134a, HFA227ea and HFA152; a surfactant such as SPAN85, Oleic acid and Lecithin; and an antioxidant such as ascorbic acid and EDTA.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A formulation suitable for inhalation, the formulation comprising at least one cannabinoid and at least one additional therapeutic agent selected from the group consisting of a beta-2 agonist and a muscarinic antagonist.

2. The formulation of claim 1, wherein the formulation comprises a beta-2 agonist and a muscarinic antagonist.

3. The formulation of claim 1, wherein the at least one cannabinoid is a member of a class selected from the group consisting of cannabichromenes, cannabicyclols, cannabidiols, cannabielsoins, cannabigerols, cannabinols, cannabinodiols, cannabitriols, cannabichromanones, isocannabinoids, and any stereoisomers, ethers, derivatives, metabolites, esters, salts, or carbon chain homologs thereof.

4. The formulation of claim 1, wherein the formulation does not comprise delta-9-tetrahydrocannabinol.

5. The formulation of claim 1, wherein the formulation does not comprise a corticosteroid.

6. The formulation of claim 1, wherein the formulation comprises at least one cannabinoid selected from the group consisting of cannabidiol, cannabigerol, cannabinol, cannabidivarin, cannabichromene, and any stereoisomers, ethers, derivatives, metabolites, esters, salts, or carbon chain homologs thereof.

7. The formulation of claim 1, comprising a short-acting beta-2 agonist selected from the group consisting of bitolterol, fenoterol, isoproterenol, levalbuterol, metaproterenol, mabuterol, pirbuterol, procaterol, ritodrine, albuterol, and terbutaline.

8. The formulation of claim 1, comprising a long-acting beta-2 agonist selected from the group consisting of arformoterol, bambuterol, clenbuterol, formoterol, salmeterol, abediterol, carmoterol, indacaterol, olodaterol, and vilanterol.

9. The formulation of claim 1, comprising a muscarinic antagonist selected from the group consisting of tiotropium, ipratropium, glycopyrronium, oxitropium, aclidinium, trospium, umeclidinium, and salts thereof.

10. The formulation of claim 1, further comprising a flavor component.

11. The formulation of claim 1, further comprising a cough suppressant.

12. A pressurized metered dose inhaler (pMDI) formulation comprising:
at least one cannabinoid;
at least one additional therapeutic agent selected from the group consisting of a beta-2 agonist and a muscarinic antagonist;
a solvent; and
a propellant.

13. The pMDI formulation of claim 12, comprising at least one cannabinoid selected from the group consisting of cannabidiol, cannabigerol, cannabinol, cannabidivarin, cannabichromene, and any stereoisomers, ethers, derivatives, metabolites, esters, salts, or carbon chain homologs thereof.

14. The pMDI formulation of claim 12, wherein the formulation does not include delta-9-tetrahydrocannabinol.

15. The pMDI formulation of claim 12, wherein the formulation does not comprise a corticosteroid.

16. The pMDI formulation of claim 12, comprising a short-acting beta-2 agonist selected from the group consisting of bitolterol, fenoterol, isoproterenol, levalbuterol, metaproterenol, mabuterol, pirbuterol, procaterol, ritodrine, albuterol, and terbutaline.

17. The pMDI formulation of claim 12, comprising a long-acting beta-2 agonist selected from the group consisting of arformoterol, bambuterol, clenbuterol, formoterol, salmeterol, abediterol, carmoterol, indacaterol, olodaterol, and vilanterol.

18. The pMDI formulation of claim 12, wherein the solvent is selected from the group consisting of water, buffered water, ethanol, propylene glycol, polyethylene glycol, erythritol, xylitol, mannitol, sorbitol, diethylene glycol monoethyl ether (Transcutol), and glycerol.

19. The pMDI formulation of claim 12, wherein the propellant is selected from the group consisting of HFA 134a, HFA 227ea, HFA-152a, and HFA-32.

20. The pMDI formulation of claim 12, further comprising a surfactant.

21. The pMDI formulation of claim 20, wherein the surfactant is selected from the group consisting of Span® 85, polyvinylpyrrolidone, oleic acid, sunflower oil, lecithin, phosphatidylcholine, isopropyl myristate, stearic acid, medium and long chain triglycerides, polysorbate 20, polysorbate 80, and sorbitan trioleate.

22. A dry powder formulation comprising:
at least one cannabinoid;
at least one additional therapeutic agent selected from the group consisting of a beta-2 agonist and a muscarinic antagonist; and
an excipient;
wherein the dry powder formulation is a dry powder suitable for inhalation.

23. The dry powder formulation of claim 22, comprising at least one cannabinoid selected from the group consisting of cannabidiol, cannabigerol, cannabinol, cannabidivarin, cannabichromene, and any stereoisomers, ethers, derivatives, metabolites, esters, salts, or carbon chain homologs thereof.

24. The dry powder formulation of claim 22, wherein the formulation does not include any psychoactive cannabinoid.

25. The dry powder formulation of claim 22, wherein the formulation does not comprise a corticosteroid.

26. The dry powder formulation of, comprising a short-acting beta-2 agonist selected from the group consisting of bitolterol, fenoterol, isoproterenol, levalbuterol, metaproterenol, mabuterol, pirbuterol, procaterol, ritodrine, albuterol, and terbutaline.

27. The dry powder formulation of claim 22, comprising a long-acting beta-2 agonist selected from the group consisting of arformoterol, bambuterol, clenbuterol, formoterol, salmeterol, abediterol, carmoterol, indacaterol, olodaterol, and vilanterol.

28. The dry powder formulation of claim 22, comprising a muscarinic antagonist selected from the group consisting of tiotropium, ipratropium, glycopyrronium, oxitropium, aclidinium, trospium, umeclidinium, and salts thereof.

29. The dry powder formulation of claim 22, wherein the excipient is selected from the group consisting of an inhalable sugar, a cellulose, a glycol, and an amino acid.
